(19) United States
(12) Patent Application Publication
(10) Pub. No.: US 2025/0325558 A1
(43) Pub. Date: Oct. 23, 2025

(54) MODULATION OF BACE1 AS A THERAPY FOR SPINOCEREBELLAR ATAXIA

(71) Applicants: Alan Jerome FOWLER, Boston, MA (US); Rudolph E. TANZI, Milton, MA (US); The General Hospital Corporation

(72) Inventors: Jaehong Suh, Lexington, MA (US); Alan Jerome Fowler, Boston, MA (US); Rudolph E. Tanzi, Milton, MA (US)

(21) Appl. No.: 18/868,526

(22) PCT Filed: May 25, 2023
(86) PCT No.: PCT/US2023/023613

§ 371 Date: Nov. 22, 2024

Related U.S. Application Data
(60) Provisional application No. 63/345,895, filed on May 25, 2022.

(51) Int. Cl. A61K 31/549; A61K 31/366; A61K 31/4025; A61K 31/541; A61K 31/704

(52) U.S. Cl. CPC A61K 31/549; A61K 31/366; A61K 31/4025; A61K 31/541; A61K 31/704

(57) ABSTRACT

Described herein are methods and compositions for treating neurodegenerative diseases including Spinocerebellar Ataxia comprising administering a BACE1 inhibitor.

### CLAIM OF PRIORITY

This application claims the benefit of U.S. Provisional Application Ser. No. 63/345,895, filed on May 25, 2022. The entire contents of the foregoing are incorporated herein by reference.

### FEDERALLY SPONSORED RESEARCH OR DEVELOPMENT

This invention was made with Government support under Grant No. AG056775 awarded by the National Institutes of Health. The Government has certain rights in the invention.

### TECHNICAL FIELD

Described herein are methods and compositions for treating neurodegenerative diseases including Spinocerebellar Ataxia comprising administering a BACE1 inhibitor.

### BACKGROUND

Spinocerebellar ataxia type 1 (SCA1) is a progressive neurodegenerative disease that impairs motor coordination and cognitive function, leading to early lethality. Expansion of CAG trinucleotide repeat that encodes a polyglutamine (polyQ) track in ataxin-1 gene (ATXN1) is the genetic determinant of this disease, which has no effective therapy.

### SUMMARY

Provided herein are methods for treating a subject who has a neurodegenerative condition associated with loss of motor function. The methods comprise administering a therapeutically effective amount of an inhibitor of BACE1. Also provided herein is an inhibitor of BACE1 for use in a method of treating a subject who has a neurodegenerative condition associated with loss of motor function

In some embodiments, the inhibitor of BACE1 is a small molecule inhibitor of BACE1, e.g., selected from the group consisting of verubecestat, MBI-10, MBI-1, MBI-3, MBI-5, MBI-9, LY2886721, LY2811376, LY3202626, elenbecestat, RG7129, TAK-070, CTS-21166, lanabecestat, AZ4217, HPP854, ginsenoside Rg1, BI 1181181, hispidin, TDC (CID 5811533), umibecestat, Monacolin K, PF-05297909, PF-06751979, CTS2116, atabecestat, RG7129 (RO5508887), SCH 1359113, a spirocyclic inhibitor (R)-50), or a fluorine-substituted 1,3-oxazine.

In some embodiments, the inhibitor of BACE1 is an antibody that binds to BACE1, optionally a bispecific antibody or a camelid antibody that binds and inhibits BACE1.

In some embodiments, the inhibitor of BACE1 is an inhibitory oligonucleotide targeting BACE1 that decreases BACE1 expression, e.g., an antisense RNA, antisense DNA, chimeric antisense oligonucleotides, antisense oligonucleotides comprising modified linkages, interference RNA (RNAi), short interfering RNA (siRNA); or a short, hairpin RNA (shRNA), or other inhibitory nucleic acid as described herein. In some embodiments, the oligonucleotide is 15 to 21 nucleotides in length. In some embodiments, at least one nucleotide of the oligonucleotide is a nucleotide analogue. In some embodiments, the oligonucleotide is a locked nucleic acid (LNA), gapmer, or mixmer.

In some embodiments, the neurodegenerative condition is a progressive loss of motor function and coordination. In some embodiments, the condition is spinocerebellar ataxia (SCA), e.g., a polyglutamine SCA, e.g., SCA1, SCA 2, SCA3, SCA6, SCA7, or SCA17, or a non-polyglutamine SCA, e.g., SCA4, SCA5, SCA8, SCA9, SCA10, and SCA11 to SCA48. In some embodiments, the condition is Friedreich's ataxia or ataxia telangiectasia. In some embodiments, the condition is Huntington's disease spinobulbar muscular atrophy, dentatorubral pallidoluysian atrophy (DRPLA), Parkinson's disease (PD), amyotrophic lateral sclerosis (ALS). In some embodiments, the subject does not have Alzheimer's disease and other tauopathies such as frontotemporal dementia, frontotemporal dementia with Parkinsonism, frontotemporal lobe dementia, multiple system tauopathy, multiple system tauopathy with presenile dementia, Wilhelmsen-Lynch disease, disinhibition-dementia-parkinsonism-amytrophy complex, Pick's disease, or Pick's disease-like dementia, corticobasal degeneration, frontal temporal dementia, Parkinson's disease, Huntington's disease, amyotrophic lateral sclerosis (ALS), multiple sclerosis, Friedreich's ataxia, Lewy body disease, spinal muscular atrophy, parkinsonism linked to chromosome 17, amyloidopathies, vascular dementia with amyloid, or cerebral amyloid angiopathy.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1. Effects of ataxin-1 loss-of-function or polyQ-expansion mutation on BACE1 expression in the brain.

FIG. 2. Increase of BACE1 expression near Aβ plaques in AD brain and in cerebellum of SCA1 mice. Upper panel: 9-month-old APP-PSI AD mouse brain. Bar=100 μm. Lower: age-dependent increase of BACE1 immunoreactivity in cerebellar molecular layer (*). Suh et al., 2019.

FIGS. 3A-C. Reduction of BACE1 expression improves locomotive activity and explorative behavior of SCA1 mice. (A) Open field trajectories of 8-month-old mice. (B) Left: total distance moved for 15 minutes. Right: time spent in the center area. Dots in each bar represent values of individual mice. (C) Rearing number in cages. Littermates were used for all behavioral tests. Values are mean±SEM. Differences between groups were analyzed using ANOVA followed by Tukey's test. *p<0.05, **p<0.01, and ***p<0.001, in all figures.

FIGS. 4A-B. Attenuated deficits of motor coordination in Atxn1^{154Q/+}; Bace1^{+/-} mice. (A) Rotarod test of 2-4, 6, and 8 month-old mice. Tests were performed in three consecutive days (D1, D2, D3), 3 times per day. *, WT vs. Atxn1^{154Q/+}. ^{#}, Atxn1^{154Q/+} vs. Atxn1^{154Q/+}; Bace1^{+/-}. ^{$,} Bace1^{+/-} vs. Atxn1^{154Q/+}. ^{&}, WT: D1 vs. D3. (B) Beam walk test of 9 month-old mice on 12 mm- and 6 mm-wide rectangular 1 meter-long beams. Some SCA1 mice reached maximum length of trial, 60 sec. Two trials per day. Differences among mouse groups were analyzed using ANOVA followed by Tukey's test for multiple comparison.

FIGS. 5A-C. BACE1 reduction in Atxn1^{154Q/+} mice. (A) BACE1 immunoreactivity in the cortex and hippocampus (upper panel) and cerebellum (lower) of 9.5-month-old WT, Atxn1^{154Q/+}, and Atxn1^{154Q/+}; Bace1^{+/-} mice. Arrows, mossy fiber ends near CA2. *, molecular layer. (B,C) Protein expression levels in cerebellum. Arrowhead: position of 154Q ataxin-1 (not detected here due to aggregation). WT, relative value of 100. Values are mean±SEM, n=5-6. *p<0.05, **p<0.01, ***p<0.001, t test.

FIGS. 6A-C. Altered expression of BACE1 and PCP in the cerebellum of SCA1 mouse. (A) BACE1 and PCP4 immunoreactivities. PC (arrow), Purkinje layer GL, granular layer. ML, molecular layer. Arrows, PCP4-positive basket cells. (B) High level BACE1 expression in soma of Purkinje neurons (arrow). (C) Count of PCP4-positive basket cells in molecular layer.

FIGS. 7A-C. Attenuated degeneration of hippocampal CA2 neurons after BACE1 reduction. (A) Upper panel: NeuN-immunolabeled hippocampal neurons around CA2 area. CA2 neurons are spares and the layer is severe curvature in SCA1 (dashed-box), which is attenuated in BACE1-reduced mice. Lower: selective PCP4 expression in CA2 neurons. (B) Quantification of PCP4 immunoreactivity in hippocampal CA2. Number of mice analyzed, n=5-6. 2-3 sections/mouse. (C) CA2 regions immunolabeled for ataxin-1 and BACE1.

FIGS. 8A-B. BACE1 reduction attenuates neurodegeneration in cortex. (A) NeuN-positive neurons in layer II/III (upper panel) and layer V (lower) of frontal motor cortex. Insets, enlarged images of dashed area, showing smaller neuronal size in SCA1 mice. Circles, showing diffused neuronal cell boundary and lack of primary dendrites in SCA1. (B) Quantification neuronal size (left) and area covered by neurons in layer II/III (right). n, number of brain sections analyzed. 1-2 sections/mouse. 4-6 mice per genotype.

FIGS. 9A-B. Partial rescue of hippocampal neurogenesis by reducing BACE1 level in SCA1 mice. (A) DCX immunoreactivity in upper dentate gyrus. (B) Counts of total (top) and projecting (bottom) DCX⁺ neurons in dentate gyrus. Number of brain sections analyzed, n=9-12. 2-3 mice per each genotype. White arrows, projecting DCX neurons. *p<0.05, ***p<0.001.

FIGS. 10A-B. (A) BACE1 and synaptoporin expression levels in SCA1 patient postmortem brains. (B) Synaptoporin expression in mouse cerebellar molecular layer.

FIGS. 11A-B. Expression patterns of NeuN, BACE1, Sez6, and Sez6L in mouse motor cortex. (A) Like Sez6 and Sez6L, BACE1 expression is detected in the soma of cortical layer V neurons. (B) Overlapping BACE1 and Sez6L expression was seen in the soma of Purkinje cells (arrows).

FIGS. 12A-B. Increase of BACE1 cleavage of Sez6 and Sez6L in Atxn1^{154Q/+} brains. (A) Left panel: Western blot analysis of Sez6 and Sez6L processing in WT, Bace1^{-/-} Bace1^{+/-} mouse brains. Secreted forms (sSez6 and sSez6L) are measured in TBS-soluble fraction of brain lysates and membrane-bound full-length forms (Sez6-fl and Sez6L-fl) are measured in RIPA-soluble brain membrane fraction. Right: Changes of processing in Atxn1^{154Q/+} and Atxn1^{154Q/+}; Bace1^{+/-} mouse cortex. (B) Densitometric quantification of protein levels in cortex. Number in bar denotes analyzed mice number.

FIGS. 13A-C. Ultrastructure analysis of hippocampal mossy fiber terminal area near CA2. (A) Enlarged proximal dendrites (arrows) of CA2 neurons in SCA1 mice. Upper, toluidine blue staining of EM section. Lower, EM image near CA2 neurons. n, nucleus. (B) Mossy fiber terminal areas. Circle: presynaptic terminals. Lower panel: high-resolution of dashed area. Blue colored, presynaptic terminals filled with vesicles. Arrows, postsynaptic density. (C) Size of presynaptic terminals. EM sections from one WT and two SCA1 mice, 50-100 synapses/mouse were analyzed.

FIGS. 14A-C. Enlarged presynaptic terminals in the cerebellar molecular layer of SCA1 mice. (A) Left: Golgi-stained Purkinje cell showing dendritic arborization and spine density heatmap. (B) EM image of dendrite (dashed outline) and spine head in molecular layer. Blue area, axonal terminal. m, mitochondria. (C) Size of presynaptic terminal and postsynaptic spine. Number of synapse analyzed 50-100/mouse. One WT and two SCA1 mice analyzed. Right: representative EM images. Arrow, synapse.

### DETAILED DESCRIPTION

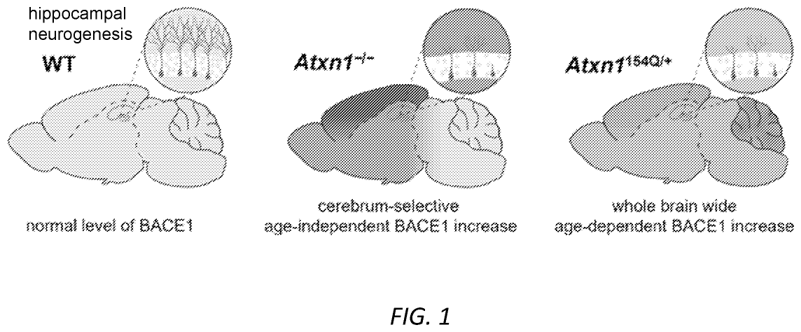

BACE1 is a key protease in Alzheimer's disease (AD) pathogenesis as it cleaves amyloid precursor protein and generate amyloid-beta (AB), the main culprit of senile plaques in AD brain. For this reason, BACE1 has been a major therapeutic target for the disease; however, recent clinical trials of BACE1 inhibitors did not produce positive outcome for AD patients. While BACE1 level in AD brain is distinctively increased in dystrophic neurites around Aβ plaques, in healthy brain, BACE1 expression is detected throughout the neuron and its proteolytic activity is involved in a variety of physiological functions including synaptic plasticity and motor coordination. During a recent study to identify ataxin-1's role in BACE1 expression and AD pathogenesis, the present inventors showed that SCA1-causing CAG repeat expansion mutation increases BACE1 expression in Atxn1^{154Q/+} mice in a disease progression dependent manner (FIG. 1 and Suh et al., 2019). BACE1 increase (30-50%) was observed throughout the brain particularly in synapse-dense areas. However, a causal role for BACE1 in disease progression or pathology was not demonstrated.

These findings, together with the observation shown herein of modest BACE1 increase in postmortem brains of SCA1 (see Example 5), prompted the present inventors to hypothesize that elevated BACE1 expression exacerbates SCA1 pathogenesis. As shown herein, BACE1 genetic reduction (Bace1^{+/-}) significantly attenuated motor deficits, neurodegeneration, and impaired hippocampal neurogenesis of Atxn1^{154Q/+} SCA1 mice. The BACE1 reduction also decreased the cleavages of Sez6 and Sez6L1, two prominent BACE1 substrates that are associated with motor activity and coordination. Furthermore, we found the presynaptic terminals in the hippocampus and cerebellum of SCA1 mice were markedly enlarged.

### Bace1's Role in Motor Function and Coordination

Among the many physiological functions in which BACE1 is involved (Das & Yan, 2019), accumulating evidence from recent studies shows a critical role in locomotive activity and motor coordination. Mice deficient of BACE1 either in whole body or in forebrain displayed increased locomotive activity with low anxiety (Laird et al, 2005; Ou-Yang et al, 2018). Concordant with this, BACE1 inhibitor-treated mice exhibited increased locomotive activity in Sez6 family protein dependent manner (Nash et al, 2021). Lack of certain BACE1 substrate proteins also caused defects in motor functions. Mice lacking either Sez6 or Sez6L showed deficits in motor coordination and cognitive function (Gunnersen et al, 2007; Nash et al, 2020; Ong-Palsson et al, 2022), and decreased cleavage of neuregulin 1 (Nrg1) by BACE1 genetic depletion or pharmacological inhibition impaired muscle spindle formation and motor coordination (Cheret et al, 2013). Mice lacking either APP or APLP2 also displayed deficits in motor functions. Lastly, increased incidence of falls observed in the clinical trials with (high dose) BACE1 inhibitor for AD patients also suggest the proteolytic function of BACE1 plays a role in regulating motor function and coordination in humans (Egan et al, 2019b).

Combining this accumulating evidence of BACE1's role in motor functions together with the inventors' findings of increased BACE1 expression in SCA1 mouse brain, the present inventors hypothesized that elevated BACE1 plays an important role in the motor deficits of SCA1.

BACE1 inhibition as a therapeutic target in SCA: Recent several phase II or III AD clinical trials with different BACE1 inhibitors have failed, as they did not produce benefits in the patients but rather adverse effects including mild cognitive worsening. The side effect on cognition was not progressive but reversible after stopping the drug administration (Hampel et al, 2021; McDade et al, 2021). Beyond the long-standing argument regarding Aβ as a viable therapeutic target for AD, one compelling possibility that could explain the failure and the side effect on cognition is that the doses of BACE1 inhibitors used in those clinical studies were too high. Near complete inhibition of BACE1 would substantially interfere with the processing of BACE1 substrates that are important for cognitive and motor functions. In support of this, in preclinical studies, BACE1 inhibitor doses that are equivalent to those used in the clinical studies almost completely reduced the cleavages of BACE1 substrates (e.g. Sez6/Sez6L), in a comparable level observed in BACE1 KO mice (Cheret et al., 2013; Nash et al., 2021). Robust decreases in BACE1 substrate cleavage were also observed in the CSF samples of BACE1 inhibitor-treated AD patients. Substantially down-regulated BACE1 substrate processing may have caused the impaired synaptic plasticity in mice and cognitive worsening in AD patients (Hampel et al, 2020).

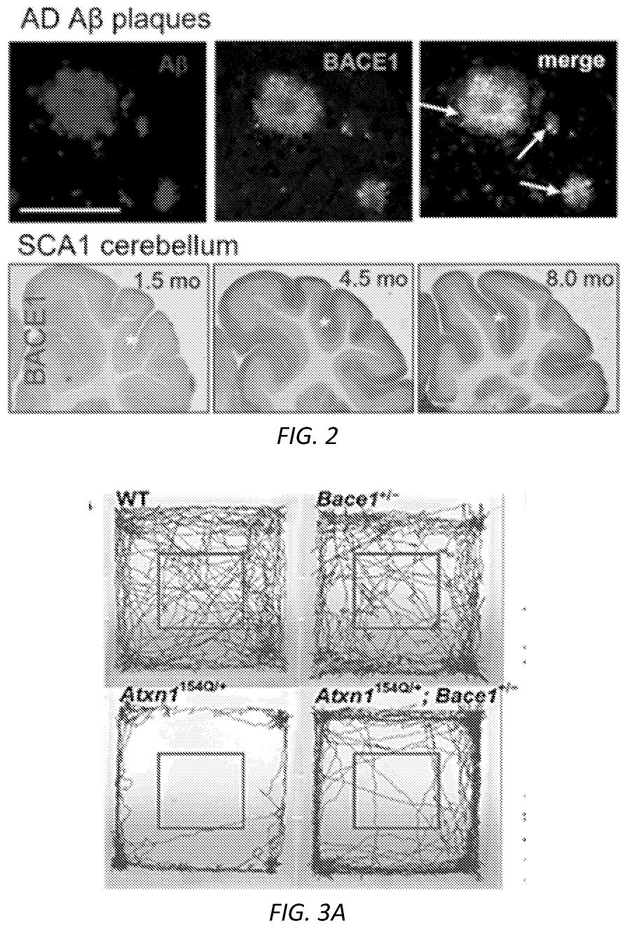

While BACE1 expression is increased both in AD and SCA1 mouse brains, there is one remarkable difference: spatial distribution. In AD brains, BACE1 elevation is limited to dystrophic neurites that surround Aβ plaques (FIG. 2 and Suh et al., 2019). However, in SCA1 mouse brains, BACE1 increase occurs throughout the brain (FIG. 2 and Suh et al., 2019). This distinct difference makes the range elevated BACE1 would affect different: focal (AD) vs. global (SCA1). In addition, we hypothesized that the required level of BACE1 inhibition would be different for the two diseases. For AD, high level BACE1 inhibition would be required to achieve maximum Aβ reduction. However, for SCA1, as shown herein partial BACE1 inhibition is sufficient to decrease the cleavage of physiological BACE1 substrate in the brain and provide therapeutic efficacy.

Concordant with this, as shown herein, partial reduction of BACE1 expression in heterozygous knockout mice (BACE1^{-/-}) produced a significant impact on motor and pathological phenotypes in SCA1 mice (see Examples 3-5). The BACE1 haploinsufficiency is comparable to partial BACE1 inhibition that would be achieved by administration of lower doses of a BACE1 inhibitor. Based on the results shown herein from BACE1 genetic reduction, administration of a low dose of BACE inhibitor that produces a reduced BACE1 enzyme activity equivalent to BACE1 haploinsufficiency, can be used in neurodegenerative conditions associated with loss of motor function, such as SCA1. Given the core motor phenotypes and brain pathology of other SCA types, either CAG repeat expansion (e.g., SCA2, SCA3, SCA6, SCA7, and SCA17) or non-CAG repeat expansion (all the others), are similar to those of SCA1, low dose of BACE1 inhibitor that would partially reduce BACE1 enzyme activity can be used to treat all SCA types. If BACE1 activity is 50% decreased, comparable to genetic haploinsufficiency, Abeta level would likely only be decreased by about 10 or 20%.

Without wishing to be bound by theory, based on the present data, partial inhibition of BACE1 is expected to be effective for SCA by restoring the BACE1 substrate cleavages that are important in regulating motor functions back to normal range. Partial BACE1 inhibition would not cause severe side effects including cognitive worsening, allowing for chronic administration as the disease progresses slowly.

### Spinocerebellar Atrophy (SCA) and Other Neurodegenerative Diseases

Spinocerebellar ataxia (SCA) is autosomal dominant neurodegenerative disease that impairs coordinated movement of the affected. Thus far over 40 different subtypes are identified based on the respective genes that harbor the causative mutations. In addition to the cerebellar pathology, common symptoms include deteriorated coordination and balance, slurred speech, and difficulty in swallowing and breathing that leads to premature death. SCA incidence rate is 1-5 (2.7) per 100,000, but there is no approved drug yet to treat or delay the disease onset. SCA1 is one of the six more common subtypes (SCA1, SCA2, SCA3, SCA6, SCA7, and SCA17) that are caused by CAG trinucleotide repeat expansion mutations within coding regions (Orr et al, 1993) (Klockgether et al, 2019; Mundwiler & Shakkottai, 2018; Paulson et al, 2017). More than 39 uninterrupted CAG repeats-encoding a polyglutamine (polyQ) track—in ataxin-1 gene (ATXN1) cause SCA1, and a longer CAG repeat is correlated with earlier disease onset and more severe prognosis (Tejwani & Lim, 2020; Zoghbi & Orr, 2000). SCA1 is pathologically characterized by cerebellar atrophy and Purkinje cell loss, and cognitive impairment is a common comorbidity in advanced stage. BACE1 inhibition can be used to ameliorate motor deficits and neuropathology of the group of polyQ SCAs, given their shared pathogenic mechanisms and core pathology (e.g., SCA1, SCA2, SCA3, SCA6, SCA7, and SCA17, caused by CAG trinucleotide repeat expansion mutations within coding regions), as well as other conditions as described herein.

Beyond SCA1, alterations in the ATXN1 gene are associated with other neurologic disorders. Deletion in chromosome 6p22 region including ATXN1 causes developmental delay and intellectual disabilities (Baroy et al, 2013; Celestino-Soper et al, 2012; Di Benedetto et al, 2013). The causative role of ataxin-1 loss in the developmental disorder was replicated in mice lacking either ataxin-1 family proteins (ATXN1 and ATXN1L) or their cellular biding partner, CIC (Lu et al, 2017). Following the genetic findings that ATXN1 is associated with AD (Bertram et al, 2008; Bettens et al, 2010), we demonstrated loss of ataxin-1 (Atxn1^{-/-}) reduces the CIC-ETV4/5-mediated inhibition of BACE1 transcription, selectively in AD-vulnerable cerebrum (Suh et al., 2019; FIG. 1). The increased BACE1 expression in turn enhanced amyloidogenic cleavage of APP and exacerbated Aβ pathology in AD mice. Elevated BACE1 levels also impaired hippocampal neurogenesis and olfactory axonal targeting. In the same study, we discovered that polyQ-expanded mutant ataxin-1 also leads to the increase of BACE1 expression in a well-characterized SCA1 mouse model (Atxn1^{154Q/+}; Suh et al., 2019). In contrast to the consequence of ataxin-1 loss-of-function, the BACE1 increase in SCA1 brain was post-transcriptionally regulated and detected both in the cerebrum and cerebellum in a disease progression-dependent manner (Suh et al., 2019).

There are other type of ataxias, such as Friedreich's ataxia and ataxia telangiectasia, which are inherited autosomal recessively. Primary symptoms of these ataxias are similar to those of SCA (e.g., lack of balance, slurred speech, difficulty in swallowing and breeding), because the cerebellum that controls muscle coordination for those functions is damaged in those ataxias as well. Given that BACE1 plays an important role in motor function and coordination and that BACE1 levels are increased in the cerebellum of SCA1 brain in a disease progression dependent manner, BACE1 modulation can be used as a therapeutic target for the non-SCA type ataxias.

Like SCA1 and other polyglutamine SCAs, Huntington's disease (HD), spinal and bulbar muscular atrophy (SBMA), and dentatorubral pallidoluysian atrophy (DRPLA) are adult-onset dominantly transmitted neurodegenerative diseases that are caused by CAG trinucleotide DNA expansion. Given that one of the major symptoms of HD, SBMA, and DRPLA is abnormal movement (e.g., chorea), modulation of BACE1 could be a potential therapy particularly for the motor symptoms of HD or DRPLA. To our knowledge, there is no study done yet to examine BACE1 level changes in the brain of HD or DRPLA.

Given BACE1 plays an important role in motor control and coordination, modulation of BACE1 could also be a therapeutic target for other movement neurodegenerative diseases, such as Parkinson's disease (PD) and amyotrophic lateral sclerosis (ALS).

Thus provided herein are methods for treating a subject who has a neurodegenerative disease that include administering to the subject an effective amount of a BACE1 inhibitor. In some embodiments, the neurodegenerative disease is spinocerebellar ataxia (SCA), including polyglutamine SCAs that are consisted of SCA1, SCA 2, SCA3, SCA6, SCA7, or SCA17. The methods can also be used to treat non-polyglutamine SCAs, e.g., SCA4, SCA5, SCA8, SCA9, SCA10, and SCA11 to SCA48 (numerically increasing subtypes), as well as Friedreich's ataxia and ataxia telangiectasia, Huntington's disease, spinobulbar muscular atrophy (SBMA), dentatorubral pallidoluysian atrophy (DRPLA), Parkinson's disease (PD), and amyotrophic lateral sclerosis (ALS).

In some embodiments, the subject has spinocerebellar ataxia (e.g., type 1, type 2, type 3 (also referred to as Machado-Joseph disease), type 6, type 7, and type 17)). In some embodiments, the subject has spinocerebellar ataxia type 1 (SCA1).

In some embodiments, the subject does not have Alzheimer's disease or another tauopathy such as frontotemporal dementia, frontotemporal dementia with Parkinsonism, frontotemporal lobe dementia, multiple system tauopathy, multiple system tauopathy with presenile dementia, Wilhelmsen-Lynch disease, disinhibition-dementia-parkinsonism-amytrophy complex, Pick's disease, or Pick's disease-like dementia, corticobasal degeneration, frontal temporal dementia, Parkinson's disease, Huntington's disease, amyotrophic lateral sclerosis (ALS), multiple sclerosis, Friedreich's ataxia, Lewy body disease, spinal muscular atrophy, parkinsonism linked to chromosome 17, amyloidopathies, vascular dementia with amyloid, or cerebral amyloid angiopathy.

As used herein, a “therapeutically effective amount” is an amount sufficient for reducing signs or symptoms of a disease, reducing (slowing) progression of a disease, reducing severity of a disease, in a subject diagnosed with the disease. A “prophylactically effective amount” is an amount that reduces the incidence or risk of a sign or symptom of a disease in a subject at risk for the disease, or delays onset of sign or symptom of the disease in a subject who is at risk, e.g., who has a genetic mutation associated with a disease as described herein. A sign or symptom can include coordination and balance (ataxia), speech and swallowing difficulties, muscle stiffness (spasticity), and weakness in the muscles that control eye movement (ophthalmoplegia). A subject as described herein can be a human, who has been diagnosed with a neurodegenerative disease as described herein, or as having a mutation associated with a neurodegenerative disease as described herein.

The present methods can include administering an amount of a BACE1 inhibitor sufficient to result in 10, 12, 15, 20, 25, 30, 35, 40, or up to about 45 or 50 or 55% inhibition of BACE1 activity (as used herein, “about” means plus or minus 10%). Methods for determining such a dose are known in the art. For example, methods for measuring and determining BACE1 activity can include measuring cleavage activity on a known substrate; BACE1 enzyme activity assays are commercially available, including those designed for BACE1 inhibitor screening, based on fluorescence resonance energy transfer (FRET) where by a fluoresence signal is observed after a substrate of BACE1 is cleaved by BACE1. These methods can be used with tissues or biological fluids (e.g., blood, homogenates, or cerebrospinal fluids). See, e.g., kits available from Abcam (Product No. ab282921) and Sigma-Aldrich (Product No. CS0010)).

An effective amount can be administered in one or more administrations, applications or dosages. A therapeutically effective amount of a therapeutic compound (i.e., an effective dosage) depends on the therapeutic compounds selected. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

Dosage, toxicity and therapeutic efficacy of the therapeutic compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

### BACE1 Inhibitors

A number of BACE1 inhibitors are known in the art, including small molecules, inhibitory antibodies, and inhibitory oligonucleotides.

Small molecule BACE1 inhibitors include LY2886721, LY2811376, LY3323795, and LY3202626 (Lilly); MBI-1, MBI-3, MBI-5, MBi-9, MBi-10, Verubecestat (MK-8931) (Merck); Elenbecestat (E2609) (Eisai); RG7129 (Roche); TAK-070 (Takeda); CTS-21166 (CoMentis); Lanabecestat (AZD3293), AZD3839, and AZ4217 (AstraZeneca); Ginsenoside Rg1 (CID 441923); BI 1181181 (Boehringer Ingelheim); Hispidin (CID310013); TDC (CID 5811533); Umibecestat (CNP520) (Novartis); Monacolin K (CID 53232); PF-05297909 and PF-06751979 (Pfizer); CTS21166 (Astellas); HPP854 (High Point Pharmaceuticals); Atabecestat (JNJ-54861911); RG7129 (RO5508887) (Roche); SCH 1359113; Spirocyclic inhibitors (e.g., as described in Hunt et al., J Med Chem. 2013 Apr. 25; 56 (8): 3379-403, such as compound (R)-50); fluorine-substituted 1,3-oxazines (e.g., as described in Hilpert et al., J Med Chem. 2013 May 23; 56 (10): 3980-95, such as the CF3 substituted oxazine 89). In some embodiments the small molecule is described in Rombouts et al., (2021) Expert Opinion on Therapeutic Patents, 31:1, 25-52, or a patent or reference cited therein.

Inhibitory antibodies that target BACE1 include AAB-001 (Bapineuzumab), AAB-003 (PF-05236812), GSK933776 and LY2062430 (Solanezumab), as well as bispecific antibodies with one arm targeting BACE1 and the other recognizing transferrin receptor to boost brain penetrance (see, e.g., Yu et al., Sci Transl Med. 2011 May 25; 3 (84): 84ra44; Atwal et al., Sci Transl Med. 2011 May 25; 3 (84): 84ra43, and U.S. Pat. No. 8,772,457) and camelid antibodies that bind and inhibit BACE1 encoded by virus (see e.g., U.S. Pat. No. 8,568,717 and US20110091446).

These and other BACE1 inhibitors useful in the present methods are described in the following US Pre-Grant Publications: 20140286963; 20140275165; 20140235626; 20140228356; 20140228277; 20140186357; 20140179690; 20140112867; 20140057927; 20140051691; 20140011802; 20130289050; 20130217705; 20130210839; 20130108645; 20130105386; 20120258961; 20120245157; 20120245155; 20120245154; 20120238557; 20120237526; 20120232064; 20120214186; 20120202828; 20120202804; 20120190672; 20120172355; 20120171120; 20120148599; 20120094984; 20120093916; 20120064099; 20120015961; 20110288083; 20110237576; 20110207723; 20110158947; 20110152341; 20110152253; 20110091446; 20110071124; 20110033463; 20100317850; 20100285597; 20100273671; 20100221760; 20100144790; 20100132060; 20100093999; 20100075957; 20100063134; 20090258925; 20090209755; 20090176836; 20090162878; 20090136977; 20090081731; 20090060987; 20090042993; 20080124379; 20070224656; 20070185042; 20060216292; 20060182736; 20060178328; 20060052327; 20050196398; 20050048641; 20040248231; 20040220132; 20040162255; 20040132680; 20040063161; 20030194745; 20020159991; and 20020157122, and U.S. Pat. Nos. 8,772,457; 8,703,785; 8,568,717; 8,415,319; 8,288,354; 8,198,269; 8,183,219; 8,058,251; 7,829,694; 7,816,378; 7,618,948; 7,273,743; and 6,713,276, as well as WO2009103626, WO2010128058, WO2011020806, WO2011029803, WO2011069934, WO2011070029, WO2011138293, WO2012019966, WO2012028563, WO2012098064, WO2012104263, WO2012107371, WO2012110459, WO2012119883, WO2012126791, WO2012136603, WO2012139993, WO2012156284, WO2012163790, WO2012168164, WO2012168175, WO2013004676, WO2013041499, WO2013110622, WO2013174781, WO2014001228, WO2014114532, WO2014150331, WO2014150340 and WO2014150344, Bazzari and Bazzari, Molecules 2022, 27 (24), 8823, and Das and Yan, CNS Drugs. 2019 March; 33 (3): 251-263.

### Inhibitory Oligonucleotides Targeting BACE1

As described above, the methods can include the administration of inhibitory oligonucleotides (“oligos”) targeting BACE1 (i.e., BACE1 mRNA or DNA) that reduce BACE1 expression. Oligos useful in the present methods and compositions include antisense oligonucleotides, ribozymes, external guide sequence (EGS) oligonucleotides, siRNA compounds, single- or double-stranded RNA interference (RNAi) compounds such as siRNA compounds, molecules comprising modified bases, locked nucleic acid molecules (LNA molecules), antagomirs, peptide nucleic acid molecules (PNA molecules), mixmers, gapmers, and other oligomeric compounds or oligonucleotide mimetics that hybridize to at least a portion of BACE1 and modulate its function. In some embodiments, the oligos include antisense RNA, antisense DNA, chimeric antisense oligonucleotides, antisense oligonucleotides comprising modified linkages, interference RNA (RNAi), short interfering RNA (siRNA); or a short, hairpin RNA (shRNA); or combinations thereof. See also WO 2015/051239.

Sequences for human BACE1 are known in the art and include the following:

| | GenBank Acc. No. |
|---|---|
| Isoform | Nucleic acid |
| beta-secretase 1 isoform A preproprotein | NM_012104.6 |
| beta-secretase 1 isoform B preproprotein | NM_138972.4 |
| beta-secretase 1 isoform C preproprotein | NM_138971.4 |
| beta-secretase 1 isoform D preproprotein | NM_138973.4 |
| beta-secretase 1 isoform E | NM_001207048.3 |
| beta-secretase 1 isoform F | NM_001207049.3 |
| beta-secretase 1 isoform G | NM_001411039.1 |

Genomic sequence for human BACE1 is at NG_029372.2, range 5000 to 35558.

In some embodiments, the oligos hybridize to at least 7, 8, 9, 10, 11, 12, 13, 14, 15, 16, 17, 18, 19, 20, 21, 22, 23, 24, 25, 26, 27, 28, 29, 30 or more consecutive nucleotides of the target sequence.

In some embodiments, the methods include introducing into the cell an oligo that specifically binds, or is complementary, to BACE1. A nucleic acid that binds “specifically” binds primarily to the target, i.e., to BACE1 RNA but not to other non-target RNAs. The specificity of the nucleic acid interaction thus refers to its function (e.g., inhibiting BACE1) rather than its hybridization capacity. Oligos may exhibit nonspecific binding to other sites in the genome or other mRNAs, without interfering with binding of other regulatory proteins and without causing degradation of the non-specifically-bound RNA. Thus this nonspecific binding does not significantly affect function of other non-target RNAs and results in no significant adverse effects. These methods can be used to treat a subject, e.g., a subject at risk for neurodegeneration following acute injury or with evidence of a chronic neurodegenerative disease, by administering to the subject a composition (e.g., as described herein) comprising an oligo that binds to BACE1. Examples of BACE1 target sequences are provided above.

In some embodiments, the methods described herein include administering a composition, e.g., a sterile composition, comprising an oligo that is complementary to BACE1 sequence as described herein. Oligos for use in practicing the methods described herein can be an antisense or small interfering RNA, including but not limited to an shRNA or siRNA. In some embodiments, the oligo is a modified nucleic acid polymer (e.g., a locked nucleic acid (LNA) molecule), a gapmer, or a mixmer.

Oligos have been employed as therapeutic moieties in the treatment of disease states in animals, including humans. Oligos can be useful therapeutic modalities that can be configured to be useful in treatment regimens for the treatment of cells, tissues and animals, especially humans.

For therapeutics, an animal, preferably a human, suspected of having or being at risk of neurodegeneration is treated by administering an oligo in accordance with this disclosure. For example, in one non-limiting embodiment, the methods comprise the step of administering to the animal in need of treatment a therapeutically effective amount of an oligo as described herein.

In some embodiments, the oligos are 10 to 50, 13 to 50, or 13 to 30 nucleotides in length. One having ordinary skill in the art will appreciate that this embodies oligonucleotides having antisense (complementary) portions of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length, or any range therewithin. It is understood that non-complementary bases may be included in such oligos; for example, an oligo 30 nucleotides in length may have a portion of 15 bases that is complementary to the targeted BACE1 RNA. In some embodiments, the oligonucleotides are 15 nucleotides in length. In some embodiments, the antisense or oligonucleotide compounds of the invention are 12 or 13 to 30 nucleotides in length. One having ordinary skill in the art will appreciate that this embodies oligos having antisense (complementary) portions of 12, 13, 14, 15, 16, 17, 18, 19, 20, 21, 22, 23, 24, 25, 26, 27, 28, 29 or 30 nucleotides in length, or any range therewithin.

Preferably the oligo comprises one or more modifications comprising: a modified sugar moiety, and/or a modified internucleoside linkage, and/or a modified nucleotide and/or combinations thereof. It is not necessary for all positions in a given oligonucleotide to be uniformly modified, and in fact more than one of the modifications described herein may be incorporated in a single oligonucleotide or even at within a single nucleoside within an oligonucleotide.

In some embodiments, the oligos are chimeric oligonucleotides that contain two or more chemically distinct regions, each made up of at least one nucleotide. These oligonucleotides typically contain at least one region of modified nucleotides that confers one or more beneficial properties (such as, for example, increased nuclease resistance, increased uptake into cells, increased binding affinity for the target) and a region that is a substrate for enzymes capable of cleaving RNA:DNA or RNA:RNA hybrids. Chimeric oligos of the invention may be formed as composite structures of two or more oligonucleotides, modified oligonucleotides, oligonucleosides and/or oligonucleotide mimetics as described above. Such compounds have also been referred to in the art as hybrids or gapmers. Representative United States patents that teach the preparation of such hybrid structures comprise, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein incorporated by reference.

In some embodiments, the oligo comprises at least one nucleotide modified at the 2′ position of the sugar, most preferably a 2′-O-alkyl, 2′-O-alkyl-O-alkyl or 2′-fluoro-modified nucleotide. In other preferred embodiments, RNA modifications include 2′-fluoro, 2′-amino and 2′ O-methyl modifications on the ribose of pyrimidines, abasic residues or an inverted base at the 3′ end of the RNA. Such modifications are routinely incorporated into oligonucleotides and these oligonucleotides have been shown to have a higher Tm (i.e., higher target binding affinity) than; 2′-deoxyoligonucleotides against a given target.

A number of nucleotide and nucleoside modifications have been shown to make the oligonucleotide into which they are incorporated more resistant to nuclease digestion than the native oligodeoxynucleotide; these modified oligos survive intact for a longer time than unmodified oligonucleotides. Specific examples of modified oligonucleotides include those comprising modified backbones, for example, phosphorothioates, phosphotriesters, methyl phosphonates, short chain alkyl or cycloalkyl intersugar linkages or short chain heteroatomic or heterocyclic intersugar linkages. Most preferred are oligonucleotides with phosphorothioate backbones and those with heteroatom backbones, particularly CH₂—NH—O—CH₂, CH, ˜N(CH₃)˜O˜CH₂ (known as a methylene(methylimino) or MMI backbone], CH₂—O—N(CH₃)—CH₂, CH₂—N(CH₃)—N(CH₃)—CH₂ and O—N(CH₃)—CH₂—CH₂ backbones, wherein the native phosphodiester backbone is represented as O—P—O—CH,); amide backbones (see De Mesmaeker et al. Ace. Chem. Res. 1995, 28:366-374); morpholino backbone structures (see Summerton and Weller, U.S. Pat. No. 5,034,506); peptide nucleic acid (PNA) backbone (wherein the phosphodiester backbone of the oligonucleotide is replaced with a polyamide backbone, the nucleotides being bound directly or indirectly to the aza nitrogen atoms of the polyamide backbone, see Nielsen et al., Science 1991, 254, 1497). Phosphorus-containing linkages include, but are not limited to, phosphorothioates, chiral phosphorothioates, phosphorodithioates, phosphotriesters, aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′; see U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455, 233; 5,466,677; 5,476,925; 5,519,126; 5,536,821; 5,541,306; 5,550,111; 5,563, 253; 5,571,799; 5,587,361; and 5,625,050.

Morpholino-based oligomeric compounds are described in Dwaine A. Braasch and David R. Corey, Biochemistry, 2002, 41 (14), 4503-4510); Genesis, volume 30, issue 3, 2001; Heasman, J., Dev. Biol., 2002, 243, 209-214; Nasevicius et al., Nat. Genet., 2000, 26, 216-220; Lacerra et al., Proc. Natl. Acad. Sci., 2000, 97, 9591-9596; and U.S. Pat. No. 5,034,506, issued Jul. 23, 1991. In some embodiments, the morpholino-based oligomeric compound is a phosphorodiamidate morpholino oligomer (PMO) (e.g., as described in Iverson, Curr. Opin. Mol. Ther., 3:235-238, 2001; and Wang et al., J. Gene Med., 12:354-364, 2010; the disclosures of which are incorporated herein by reference in their entireties).

### Pharmaceutical Compositions and Methods of Administration

The methods described herein can include the administration of pharmaceutical compositions and formulations comprising BACE1 inhibitors and/or oligonucleotides designed to target BACE1.

In some embodiments, the compositions are formulated with a pharmaceutically acceptable carrier. The pharmaceutical compositions and formulations can be administered parenterally, topically, orally or by local administration, such as by aerosol or transdermally. The pharmaceutical compositions can be formulated in any way and can be administered in a variety of unit dosage forms depending upon the condition or disease and the degree of illness, the general medical condition of each patient, the resulting preferred method of administration and the like. Details on techniques for formulation and administration of pharmaceuticals are well described in the scientific and patent literature, see, e.g., Remington: The Science and Practice of Pharmacy, 21st ed., 2005.

The oligos can be administered alone or as a component of a pharmaceutical formulation (composition). The compounds may be formulated for administration, in any convenient way for use in human or veterinary medicine. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Formulations of the compositions of the invention include those suitable for intradermal, inhalation, oral/nasal, topical, parenteral, rectal, and/or intravaginal administration. The formulations may conveniently be presented in unit dosage form and may be prepared by any methods well known in the art of pharmacy. The amount of active ingredient (e.g., nucleic acid sequences of this invention) which can be combined with a carrier material to produce a single dosage form will vary depending upon the host being treated, the particular mode of administration, e.g., intradermal or inhalation. The amount of active ingredient which can be combined with a carrier material to produce a single dosage form will generally be that amount of the compound which produces a therapeutic effect, e.g., an antigen specific T cell or humoral response.

Pharmaceutical formulations of this invention can be prepared according to any method known to the art for the manufacture of pharmaceuticals. Such drugs can contain sweetening agents, flavoring agents, coloring agents and preserving agents. A formulation can be admixtured with nontoxic pharmaceutically acceptable excipients which are suitable for manufacture. Formulations may comprise one or more diluents, emulsifiers, preservatives, buffers, excipients, etc. and may be provided in such forms as liquids, powders, emulsions, lyophilized powders, sprays, creams, lotions, controlled release formulations, tablets, pills, gels, on patches, in implants, etc.

Pharmaceutical formulations for oral administration can be formulated using pharmaceutically acceptable carriers well known in the art in appropriate and suitable dosages. Such carriers enable the pharmaceuticals to be formulated in unit dosage forms as tablets, pills, powder, dragees, capsules, liquids, lozenges, gels, syrups, slurries, suspensions, etc., suitable for ingestion by the patient. Pharmaceutical preparations for oral use can be formulated as a solid excipient, optionally grinding a resulting mixture, and processing the mixture of granules, after adding suitable additional compounds, if desired, to obtain tablets or dragee cores. Suitable solid excipients are carbohydrate or protein fillers include, e.g., sugars, including lactose, sucrose, mannitol, or sorbitol; starch from corn, wheat, rice, potato, or other plants; cellulose such as methyl cellulose, hydroxypropylmethyl-cellulose, or sodium carboxy-methylcellulose; and gums including arabic and tragacanth; and proteins, e.g., gelatin and collagen. Disintegrating or solubilizing agents may be added, such as the cross-linked polyvinyl pyrrolidone, agar, alginic acid, or a salt thereof, such as sodium alginate. Push-fit capsules can contain active agents mixed with a filler or binders such as lactose or starches, lubricants such as talc or magnesium stearate, and, optionally, stabilizers. In soft capsules, the active agents can be dissolved or suspended in suitable liquids, such as fatty oils, liquid paraffin, or liquid polyethylene glycol with or without stabilizers.

Aqueous suspensions can contain an active agent (e.g., nucleic acid sequences of the invention) in admixture with excipients suitable for the manufacture of aqueous suspensions, e.g., for aqueous intradermal injections. Such excipients include a suspending agent, such as sodium carboxymethylcellulose, methylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

In some embodiments, oil-based pharmaceuticals are used for administration of nucleic acid sequences of the invention. Oil-based suspensions can be formulated by suspending an active agent in a vegetable oil, such as arachis oil, olive oil, sesame oil or coconut oil, or in a mineral oil such as liquid paraffin; or a mixture of these. See e.g., U.S. Pat. No. 5,716,928 describing using essential oils or essential oil components for increasing bioavailability and reducing inter- and intra-individual variability of orally administered hydrophobic pharmaceutical compounds (see also U.S. Pat. No. 5,858,401). The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto (1997) J. Pharmacol. Exp. Ther. 281:93-102.

Pharmaceutical formulations can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent. In alternative embodiments, these injectable oil-in-water emulsions of the invention comprise a paraffin oil, a sorbitan monooleate, an ethoxylated sorbitan monooleate and/or an ethoxylated sorbitan trioleate.

The pharmaceutical compounds can also be administered by in intranasal, intraocular and intravaginal routes including suppositories, insufflation, powders and aerosol formulations (for examples of steroid inhalants, see e.g., Rohatagi (1995) J. Clin. Pharmacol. 35:1187-1193; Tjwa (1995) Ann. Allergy Asthma Immunol. 75:107-111). Suppositories formulations can be prepared by mixing the drug with a suitable non-irritating excipient which is solid at ordinary temperatures but liquid at body temperatures and will therefore melt in the body to release the drug. Such materials are cocoa butter and polyethylene glycols.

In some embodiments, the pharmaceutical compounds can be delivered transdermally, by a topical route, formulated as applicator sticks, solutions, suspensions, emulsions, gels, creams, ointments, pastes, jellies, paints, powders, and aerosols.

In some embodiments, the pharmaceutical compounds can also be delivered as microspheres for slow release in the body. For example, microspheres can be administered via intradermal injection of drug which slowly release subcutaneously; see Rao (1995) J. Biomater Sci. Polym. Ed. 7:623-645; as biodegradable and injectable gel formulations, see, e.g., Gao (1995) Pharm. Res. 12:857-863 (1995); or, as microspheres for oral administration, see, e.g., Eyles (1997) J. Pharm. Pharmacol. 49:669-674.

In some embodiments, the pharmaceutical compounds can be parenterally administered, such as by intravenous (IV) administration or administration into a body cavity or lumen of an organ. These formulations can comprise a solution of active agent dissolved in a pharmaceutically acceptable carrier. Acceptable vehicles and solvents that can be employed are water and Ringer's solution, an isotonic sodium chloride. In addition, sterile fixed oils can be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of active agent in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated using those suitable dispersing or wetting agents and suspending agents. The sterile injectable preparation can also be a suspension in a nontoxic parenterally-acceptable diluent or solvent, such as a solution of 1,3-butanediol. The administration can be by bolus or continuous infusion (e.g., substantially uninterrupted introduction into a blood vessel for a specified period of time).

In some embodiments, the pharmaceutical compounds and formulations can be lyophilized. Stable lyophilized formulations comprising an oligo can be made by lyophilizing a solution comprising a pharmaceutical of the invention and a bulking agent, e.g., mannitol, trehalose, raffinose, and sucrose or mixtures thereof. A process for preparing a stable lyophilized formulation can include lyophilizing a solution about 2.5 mg/mL protein, about 15 mg/mL sucrose, about 19 mg/mL NaCl, and a sodium citrate buffer having a pH greater than 5.5 but less than 6.5. See, e.g., U.S. 20040028670.

The compositions and formulations can be delivered by the use of liposomes. By using liposomes, particularly where the liposome surface carries ligands specific for target cells, or are otherwise preferentially directed to a specific organ, one can focus the delivery of the active agent into target cells in vivo. See, e.g., U.S. Pat. Nos. 6,063,400; 6,007,839; Al-Muhammed (1996) J. Microencapsul. 13:293-306; Chonn (1995) Curr. Opin. Biotechnol. 6:698-708; Ostro (1989) Am. J. Hosp. Pharm. 46:1576-1587. As used in the present invention, the term “liposome” means a vesicle composed of amphiphilic lipids arranged in a bilayer or bilayers. Liposomes are unilamellar or multilamellar vesicles that have a membrane formed from a lipophilic material and an aqueous interior that contains the composition to be delivered. Cationic liposomes are positively charged liposomes that are believed to interact with negatively charged DNA molecules to form a stable complex. Liposomes that are pH-sensitive or negatively-charged are believed to entrap DNA rather than complex with it. Both cationic and noncationic liposomes have been used to deliver DNA to cells.

Liposomes can also include “sterically stabilized” liposomes, i.e., liposomes comprising one or more specialized lipids. When incorporated into liposomes, these specialized lipids result in liposomes with enhanced circulation lifetimes relative to liposomes lacking such specialized lipids. Examples of sterically stabilized liposomes are those in which part of the vesicle-forming lipid portion of the liposome comprises one or more glycolipids or is derivatized with one or more hydrophilic polymers, such as a polyethylene glycol (PEG) moiety. Liposomes and their uses are further described in U.S. Pat. No. 6,287,860.

The formulations of the invention can be administered for prophylactic and/or therapeutic treatments. In some embodiments, for therapeutic applications, compositions are administered to a subject who is at risk of or has a disorder described herein, in an amount sufficient to cure, alleviate or partially arrest the clinical manifestations of the disorder or its complications; this can be called a therapeutically effective amount.

The amount of pharmaceutical composition adequate to accomplish this is a therapeutically effective dose. The dosage schedule and amounts effective for this use, i.e., the dosing regimen, will depend upon a variety of factors, including the stage of the disease or condition, the severity of the disease or condition, the general state of the patient's health, the patient's physical status, age and the like. In calculating the dosage regimen for a patient, the mode of administration also is taken into consideration.

The dosage regimen also takes into consideration pharmacokinetics parameters well known in the art, i.e., the active agents' rate of absorption, bioavailability, metabolism, clearance, and the like (see, e.g., Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacol. 24:103-108; Remington: The Science and Practice of Pharmacy, 21st ed., 2005). The state of the art allows the clinician to determine the dosage regimen for each individual patient, active agent and disease or condition treated. Guidelines provided for similar compositions used as pharmaceuticals can be used as guidance to determine the dosage regiment, i.e., dose schedule and dosage levels, administered practicing the methods of the invention are correct and appropriate.

Single or multiple administrations of formulations can be given depending on for example: the dosage and frequency as required and tolerated by the patient, the degree and amount of therapeutic effect generated after each administration (e.g., effect on tumor size or growth), and the like. The formulations should provide a sufficient quantity of active agent to effectively treat, prevent or ameliorate conditions, diseases or symptoms.

In alternative embodiments, pharmaceutical formulations for oral administration are in a daily amount of between about 1 to 100 or more mg per kilogram of body weight per day. Lower dosages can be used, in contrast to administration orally, into the blood stream, into a body cavity or into a lumen of an organ. Substantially higher dosages can be used in topical or oral administration or administering by powders, spray or inhalation.

In some embodiments, the methods described herein can include co-administration with other drugs or pharmaceuticals, e.g., compositions for providing cholesterol homeostasis. For example, the oligos can be co-administered with drugs for treating or reducing risk of a disorder described herein.

### EXAMPLES

The invention is further described in the following examples, which do not limit the scope of the invention described in the claims.

### Materials and Methods

The following materials and methods were used in the Examples below.

Animals. Spinocerebellar ataxia type-1 154Q knock-in mice (Atxn1^{15AQ+}) on a C57BL/6 genetic background were previously described (Watase et al., 2002). BACE1 KO (Bace1^{-/-}) mice (Cai et al., 2001) on a C57BL/6 background were purchased from Jackson Laboratory. Atxn1^{15AQ+}; Bace1^{+/-} mice were generated by crossing Atxn1^{15AQ+} with Bace1^{+/-} mice. Genotypes of pups were determined by PCR of tail DNA. Mice were age- and sex-matched within experiments. Equal or similar numbers of male and female mice were used in all experiments. Ages of mice used for each experiment were described in the Results, Figures, and Figure Legends. All mouse generation, husbandry, and experimental procedures were approved by the Massachusetts General Hospital Subcommittee on Research Animal Care (MGH SRAC), and conform to the NIH guidelines of the care and use of laboratory animals. Mice were housed under specific pathogen free condition in an animal facility at MGH, under the care of full-time veterinarians or veterinarian technicians.

Behavioral testing. For each test, mice were left to habituate in the testing room with ambient noise for 30 minutes prior to testing.

Open-field: Mice were placed in a non-transparent acryl-walled open field enclosure (45×45×45 cm³). The area of the enclosure is virtually defined with a center zone surrounded by a peripheral zone. The animal's activity was video recorded for 15 minutes using an overhead camera. Locomotor activity including total distance moved (cm) and time spent within a center zone were analyzed with an automated tracking system (EthoVision XT 9.0, Noldus).

Rearing exploratory behavior: To assess exploratory behavior, mice were observed in their home cages for rearing behaviors. Video recordings of mice were analyzed to count the number of instances of weight put on hits hind legs and raise both forelimbs from the ground or place forelimbs on the wall or object within the cage and lift its head. Rears for each mouse were recorded per 60 seconds and averaged over the session.

Rotarod: To evaluate motor coordination mice were tested on an accelerating rotating rod (Ugo Basile). Mice were placed on the rotating rod (3 cm diameter, 30 cm long) in four trails every day for a period of 4 days. Each trial lasted for a maximum of 10 minutes. The rod accelerated from 4 rpm to 40 rpm over 5 minutes and remains at 40 rpm for an additional 5 minutes. The time the mice spent on the rod without falling was recorded (latency to fall). Two subsequent rotations around by holding the rod were also counted as a fall.

Balance-beam test: To evaluate motor coordination, mice were tested to traverse a 100 cm long and 6 mm or 12 mm wide beam 50 cm above the ground between a brightly illuminated starting platform and a darkened enclosed escape box at the other end. Mice were trained to walk across the beam before testing in three trails for a period of 3 days. Each trial lasted for a maximum of 120 seconds. The time (seconds) to cross for each mouse was recorded and averaged over the three trials.

Brain tissue preparation and Western blot analysis. For biochemical analysis, mice were perfused with PBS and brains were stored at −80° C. until use. If needed, each brain region from freshly removed brains was dissected out in ice-cold PBS and kept at −80° C. Mouse brain hemispheres were homogenized in TBS buffer containing 5 mM EDTA, 2 mM 1,10-phenanthroline and protease inhibitor mixture (Pierce). Homogenates were centrifuged at 100,000×g for 1 hr at 4° C. The supernatants were used for analyses of secreted proteins, while pellets extracted with RIPA buffer (1% NP40, 0.1% SDS and 0.5% sodium deoxycholate in TBS) containing protease inhibitors were used for the analysis of membrane-bound proteins. To obtain total brain lysates, including both cytosolic and membrane proteins, TBS brain lysates were homogenized in RIPA buffer containing protease inhibitors, followed by centrifugation at 13,000×g for 10 min. The RIPA-soluble supernatants were used as total protein lysates. Protein lysates were separated on 4-12% gradient NuPAGE Novex Bis-Tris SDS Midi gel (Invitrogen) and transferred to PVDF membrane (iBlot, Invitrogen). Pre-stained standard (SeeBlue Plus2, Invitrogen) was used as a size marker for western blot analysis. Antibodies used for western blotting were Ataxin-1 (76/8, NeuroMab), BACE1 (D10E5, Cell Signaling; ab2077, Abcam), calbindin, synaptoporin (102 002, Synaptic Systems), Sez6 (14E5, Abcam), Sez6L (21D5, gift from Dr. Lichtenthaler), pan-actin (MS-1295-P0, Fisher Scientific) and GAPDH (MAB374, EMD Millipore). Immunoreactive bands on blots were detected with enhanced chemiluminescence reagents (Pierce) on X-ray film. Films were scanned and band intensities of expected sizes were quantified by densitometric analysis using ImageJ (NIH).

Immunohistochemical staining of brain sections. For histological analysis, mice were perfused with PBS and the brains were fixed with 4% paraformaldehyde (PFA) for 2-3 days, followed by incubation in 30% sucrose solution for 2-3 days until the brains sunk to the bottom. For immunohistochemical or immunofluorescence staining, sucrose-downed mouse brains were sagittally sectioned by a sliding microtome at 40 μm thick, unless otherwise indicated. For the immunostaining, at least ten brain sections encompassing a whole hemisphere of each mouse were incubated with 0.3% Triton X-100, 0.3% Donkey serum and 3% H₂O₂ for 20 minutes at room temperature, to increase antibody permeability and to remove endogenous peroxidase activity. Sections were washed with TBS and then incubated with 0.3% Triton X-100, 5% Donkey serum and primary antibody for overnight at 4° C. If the primary antibodies were generated from mice, brain sections were pre-incubated with MOM Mouse Ig blocking reagent (Vector Laboratories) for 1 hr at room temperature prior to the primary antibody incubation. Primary antibodies used for immunostaining are Ataxin-1 (76/8, NeuroMab), Aβ (3D6, Eli Lilly), BACE1 (D10E5, Cell Signaling), DCX (SC-8066, Santa Cruz), NeuN (RPCA-FOX3, EnCor Biotechnology), PCP4 (HPA005792, Atlas Antibodies), synaptoporin (102 002, Synaptic Systems), Sez6 (14E5, Abcam), Sez6L (21D5, gift from Dr. Lichtenthaler). For light microscopic analysis, the bound primary antibodies were detected by biotin-conjugated secondary antibodies, followed by incubation with avidin-biotin-peroxidase complex and by subsequent development of the bound peroxidase activity with 3,3′-diaminobenzidine (DAB, Vector Laboratories). Stained sections were dehydrated with increasing concentration of ethanol and mounted with Cytoseal (Thermo Fisher Scientific). Staining was visualized with a microscope (Nikon TE300), and photomicrographs were taken with an attached digital camera and associated software (Nikon Elements). For confocal microscopic analysis, the primary antibody-bound sections were incubated with AlexaFluor 488 or 568-conjugated secondary antibody (Molecular Probes). DAPI (4′,6-diamidino-2-phenylindole, Sigma) was used for nuclear counter staining, and DABCO (1,4-diazabicyclo[2.2.2]octane, Sigma) was used for mounting. Fluorescence images were captured by confocal microscopes (Olympus IX70, Nikon C2; Micro Video Systems).

Hippocampal neurogenesis analysis. To analyze the dendritic development of newborn neurons in the hippocampus, brain sections were labeled with anti-DCX antibody and the immunoreactive signals were developed with DAB. Photomicrographs were taken with the 10× objective lens, and DCX⁺ neurons in subgranular cell layer (total DCX⁺ neurons) were counted for at least 3 brain sections per each mouse, containing full hippocampal formation. DCX⁺ neurons that have dendrites which pass through the granular neuronal layer were counted as projecting DCX⁺ neurons. To compare hippocampal neurogenesis among 5-month-old WT, Bace1^{+/-}, Atxn1^{15AQ+} and Atxn1^{15AQ+}; Bace1^{+/-} mice, ImageJ was used to quantify DCX⁺ cell bodies in the subgranular cell layer (total DCX⁺ cells). The amounts of projecting DCX⁺ neurons were determined by quantifying the areas covered by DCX-positive immunoreactivity within the granular cell layer (ImageJ).

Analysis of cortical neurodegeneration. Photomicrographs of mouse brain sections (20× images) stained for NeuN were used to count NeuN⁺ neurons and neuronal size in layers II/III and V of the motor cortex. To count the number of neurons and the area covered, an empty box of a set size that covered the layer II/III or layer V in the motor cortex was used across all images from all mice. Within the box cells were counted and size of cell bodies were measured using ImageJ. For a more accurate comparison among different mice, sagittal sections that are between 1 and 2 mm away from the brain midline were analyzed.

Electron microscopy. For ultrastructural analysis, mice were anesthetized with isoflurane and transcardially perfused with PBS followed by EM fixative solution (4% paraformaldehyde, 75 mM lysine-HCl, 10 mM sodium-periodate, and 0.15M sucrose in in phosphate buffer). Whole brains were removed and 1 mm sagittal sections were collected and further post-fixed in EM fixative solution for 4 hours at room temperature on an orbital shaker. Sagittal sections were washed in PBS and 1 mm² specimens were excised from the hippocampus, cortex, and cerebellum in PBS under a dissection microscope. Samples were immersion fixed further in 2% paraformaldehyde/2.5% glutaraldehyde in 0.1M cacodylate buffer for 2 hr at room temperature, then allowed to infiltrate further in fixative overnight at 4° C. Samples were rinsed several times in 0.1M cacodylate buffer, infiltrated 1 hr in 1% osmium tetroxide, rinsed several times again in cacodylate buffer, then dehydrated through a graded series of ethanols to 100%, followed by a brief (10 min) dehydration in 100% propylene oxide. Samples were then allowed to pre-infiltrate 2-3 hours in a 2:1 mix of propylene oxide:Eponate resin (Ted Pella, Redding, CA), then transferred into a 1:1 mix of propylene oxide:Eponate resin for overnight infiltration at room temperature on a gentle rotator. The following day, specimens were placed into a 2:1 mix of Eponate:propylene oxide, allowed to infiltrate at least 2 hours, then transferred into fresh 100% Eponate for several hours. Tissue was placed into flat molds with fresh 100% Eponate resin and allowed to polymerize in a 60° C. oven (24-48 hrs). Semi-thin (1 μm) sections were collected and stained with 0.1% toluidine blue and previewed by light microscopy to determine orientation. Ultra-thin (70 nm) sections were cut using a Leica EM UC7 ultramicrotome, collected onto formvar-coated grids, stained with 2% uranyl acetate and Reynold's lead citrate and examined in a JEOL JEM 1011 transmission electron microscope at 80 kV. To measure synaptic structures, electron micrographs were taken at 20-40,000× objective lens and analyzed using ImageJ.

Neurodegeneration analysis: To determine if BACE1 genetic reduction would attenuate neurodegeneration in SCA1, we compared neuronal loss and structural changes in motor cortex, hippocampal CA2, and cerebellar Purkinje cell layer. We employed the methods described in Suh 2019 Cell to analyze neurodegeneration in WT, Atxn1^{154Q/+}, and Atxn1^{154Q/+}; Bace1^{+/-} mice. NeuN (EnCor Biotechnology) immunolabeling was utilized for the analysis of cortical and hippocampal neuronal loss and structural changes. PCP4 (Atlas antibodies) and/or calbindin (Swant) immunostaining was utilized for Purkinje cell analysis. Four male and four female mice of each genotype were euthanized and perfused with PBS at 9 months of age. For immunohistochemical staining, each brain hemisphere was fixed with 4% paraformaldehyde (PFA) and sucrose-downed and sectioned sagittally with a sliding microtome with 40 μm thickness. At least ten sections encompassing the whole hemisphere of each mouse were incubated with 0.3% Triton X-100, 0.3% donkey serum and 3% H₂O₂ for 20 minutes at room temperature. Sections were washed with TBS and then incubated with 0.3% Triton X-100, 5% Donkey serum and a primary antibody for overnight at 4° C. For cortical neuron analysis, bound NeuN antibody in the section was detected by subsequent incubation with biotin-conjugated anti-mouse antibody, avidin-biotin-peroxidase complex and color-developed with 3,3′-diamino-benzidine (DAB, Vector Laboratories). For more accurate comparison among different mice, sagittal sections that were between 1 and 2 mm away from the brain midline, containing full hippocampal formation, were selected for analysis. NeuN-positive cell number and size are quantified by ImageJ program (NIH) using ‘Analyze Particles’ method. For hippocampal CA2 neuron analysis, brain sections were co-labeled with antibodies for NeuN and CA2 marker PCP4 and subsequently incubated with AlexaFluor 568 or 488 for confocal microscopy analysis (Suh 2019). Numbers of PCP4+ and NeuN+ neurons in CA2 were manually counted. For Purkinje neuron analysis, sections were labeled with antibodies for either PCP4 and calbindin and immunolabeled Purkinje cells were manually counted and analyzed as ‘cell number/mm-length of PC layer’ (Rousseaux et al, 2018). In cell counting analysis, experimenters are blinded for the genotypes of the samples.

Purkinje neurons electrophysiology analysis. Dysfunction of Purkinje neurons is a common and early feature of many SCA models and previous studies have shown that changes in the firing pattern of Purkinje neurons are directly related to either motor dysfunction or improvement in mouse models (Chopra et al, 2018; Hourez et al, 2011; McLoughlin et al, 2018; Scoles et al, 2017). Purkinje neurons of Atxn1^{154Q/+} mice were shown to display irregular spiking patterns at 4.5 months of age while their firing frequency was not changed (Bushart et al, 2021). In this study, we examined if BACE1 genetic reduction would rescue the abnormal firing pattern of Atxn1^{154Q/+} Purkinje neurons. Specifically, 300 μm-thick parasagittal cerebellar slices were cut using a Leica VT1000S vibratome and incubated in oxygenated artificial cerebrospinal fluid (ACSF) of the following composition (mM): NaCl 126, KCl 3.5, CaCl₂) 2.0, MgCl₂ 1.3, NaHCO₃ 25, NaH₂PO₄ 1.2 and glucose 11 (pH 7.4) at least 1 hr before use. For electrophysiological recordings, individual slices were transferred to a conventional submerged-type chamber and continuously superfused with oxygenated ACSF at 32° C. and at a rate of 2-3 ml/min. Whole-cell recordings were made using a MultiClamp 700B computer-controlled current and voltage clamp amplifier. IR-DIC microscopy (AxioScop FS, Carl Zeiss) through a 40× water immersion objective will be used for visual control of experiments and identification of Purkinje neurons from cerebellar lobules II to V (McLoughlin et al., 2018; Shakkottai et al, 2011). Patch electrodes were made from borosilicate glass capillaries (G150F-4, Warner Instrument Corporation) using the model P-97 flaming/brown micropipette puller (Sutter Instrument Company). For whole cell recording, pipettes were filled with solution containing (mM): potassium gluconate 135, CaCl₂) 0.1, MgCl₂ 2, Na₂ATP 2, EGTA 1 and HEPES 10, pH 7.25. The liquid junction potential was measured, and all voltages reported were corrected values. The electrical signals were digitized using an analog-to-digital converter Axon DigiData 1550B (Molecular Devices). pClamp and Clampfit 10.3 (Molecular Devices), Origin 2018 (Microcal Software), and SigmaPlot 11.0 (Systat Software) programs were used for data acquisition and analysis. Purkinje cells were recorded from mice in each genotype at 8 months of age. Purkinje cell firing frequency and irregularity (i.e., coefficient of variation of inter-spike interval) were analyzed and compared among different mouse genotypes.

Quantification and Statistical Analyses. All data are presented as mean±standard error of the mean (SEM). All data requiring statistical testing were analyzed using t-test (two-tailed unequal variance, Microsoft Excel) or one-way analysis of variance (ANOVA) followed by Tukey's test (PRISM). *, **, and *** denote p<0.05, p<0.01, and p<0.001, respectively, in all figures. p values <0.05 was considered statistically significant. All of the statistical details of experiments, including the statistical tests used, exact value of n, and what n represents (e.g., number of mice), can be found in the figures and figure legends.

### Example 1. Effects of Ataxin-1 Loss-of-Function or PolyQ-Expansion Mutation on BACE1 Expression in the Brain

Previously, in genome-wide association studies (GWAS), our group and others have discovered that ATXN1 is associated with AD (Bertram et al., 2008; Bettens et al., 2010). In a recent follow-up mechanism study (Suh et al, 2019), we demonstrated that loss of ataxin-1 (Atxn1^{-/-}) reduces CIC-ETV4/5-mediated inhibition of BACE1 transcription, selectively in AD-vulnerable cerebrum (FIG. 1). The increased BACE1 level in ataxin-1 KO brain enhanced amyloidogenic cleavage of APP and exacerbated Aβ pathology in AD mice. Elevated BACE1 expression also impaired hippocampal neurogenesis and olfactory axonal targeting. In the same study, it was discovered in a well-characterized SCA1 mouse model (Atxn1^{154Q/+}) that polyQ-expanded mutant ataxin-1 also leads to increase of BACE1 expression in the brain. The increase was post-transcriptionally regulated and observed both in the cerebrum and cerebellum in a disease progression-dependent manner (FIG. 1)

### Example 2. Increase of BACE1 Expression Near Aβ Plaques in AD Brain and in Cerebellum of SCA1 Mice

While BACE1 expression was increased both in AD and SCA1 mouse brains, there was one remarkable difference: spatial distribution. In AD brains, BACE1 elevation is limited to dystrophic neurites that surround Aβ plaques (FIG. 2). However, in SCA1 mouse brains, BACE1 increase occurs throughout the brain (FIG. 2). This distinct difference makes the range elevated BACE1 would affect different: focal (AD) vs. global (SCA1). In addition, the required level of BACE1 inhibition will be different for the two diseases. For AD, high level BACE1 inhibition is required to achieve maximum Aβ reduction. However, for SCA1, partial BACE1 inhibition will be sufficient to decrease the cleavage of physiological BACE1 substrate in the brain

### Example 3. Reduction of BACE1 Expression Attenuates Motor Deficits and Brain Pathology of SCA1 Mice

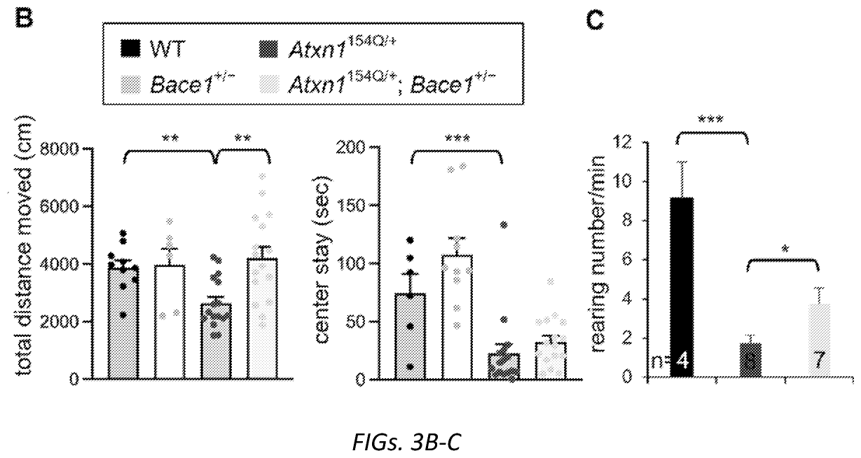

BACE1 expression is increased in Atxn1^{154Q/+} SCA1 mouse brains in a disease-dependent manner (Suh et., 2019), and recent studies have shown BACE1 is critically involved in the regulation of motor function and coordination (Cheret et al., 2013; Egan et al., 2019b; Gunnersen et al., 2007; Laird et al., 2005; Nash et al., 2020; Nash et al., 2021; Ong-Palsson et al., 2022; Ou-Yang et al., 2018). These findings led us to hypothesize that elevated BACE1 expression may affect the motor phenotypes of SCA1. To test this hypothesis, we reduced BACE1 expression in the SCA1 brain and examined if that would decrease motor impairments observed in a mouse model of SCA1, Atxn1^{154Q/+} mice. BACE1 reduction was achieved by crossing male Atxn1^{154Q/+} with female Bace1^{+/-} mice. The Atxn1^{154Q/+}; Bace1^{+/-} mice generated from the crossing were morphologically not distinguishable from Atxn1^{154Q/+} littermates; they showed slowed growth rate than WT mice and exhibited weight loss and kyphosis at several months of age. However, when assessed with behavioral tests aimed to measure ataxia severity (Guyenet et al, 2010), the majority of Atxn1^{154Q/+}; Bace1^{+/-} mice moved more actively and exploratively than Atxn1^{154Q/+} littermates, especially in new environments (e.g., new cages). Video analysis of cage behavior also revealed Atxn1^{154Q/+}; Bace1^{+/-} mice were more vigilant to the surroundings and reared more frequently than Atxn1^{154Q/+} mice. In open field tests, Atxn1^{154Q/+} mice of 6- or 8-months old showed reduced locomotive activity (˜36%) as compared to WT. In contrast, Atxn1^{154Q/+}; Bace1^{+/-} mice traveled significantly longer, at levels comparable to that of WT, while maintaining thigmotaxis (FIGS. 3A-C).

### Example 4. Attenuated Deficits of Motor Coordination in Atxn1^{154Q/+}; Bace1^{+/-} Mice

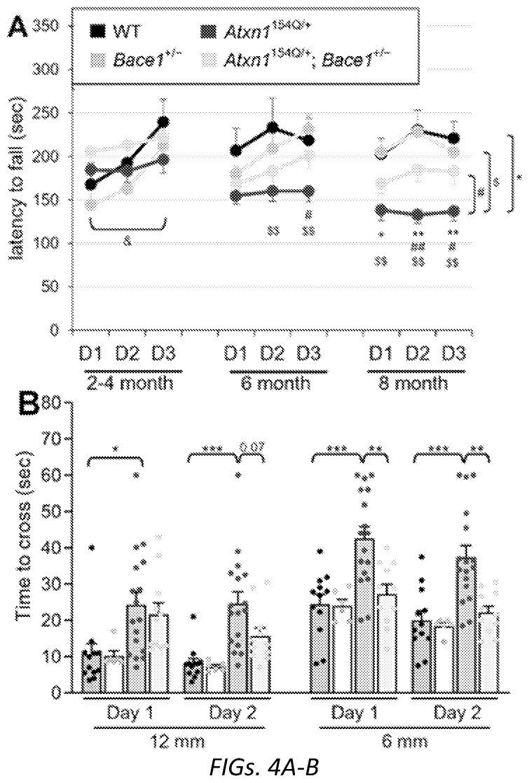

We next examined if BACE1 reduction would affect motor coordination. In contrast to WT, SCA1 (Atxn1^{154Q/+}) mice's retention time on accelerating rotarod got shorter (60% of WT) as aging up to 8 months (FIG. 4A). While the performance of Atxn1^{154Q/+}; Bace1^{+/-} mice also deteriorated as aging but significantly less (81% of WT) than Atxn1^{154Q/+} littermates. The impact of BACE1 reduction on motor behavior was most pronounced in beam walk tests. While Atxn1^{154Q/+} mice of 8-months old needed double amount of the time than WT littermates to walk across a 6 mm-wide/1-meter-long rectangular beam, Atxn1^{154Q/+}; Bace1^{+/-} littermates moved across as fast as WT mice (FIG. 4B). Together, these results from the behavioral tests suggest that reduction of BACE1 expression attenuates locomotive and coordination deficits of SCA1 mice.

### Example 5. Genetic Reduction of BACE1 Decreases Neurodegeneration in SCA1 Mouse Brain

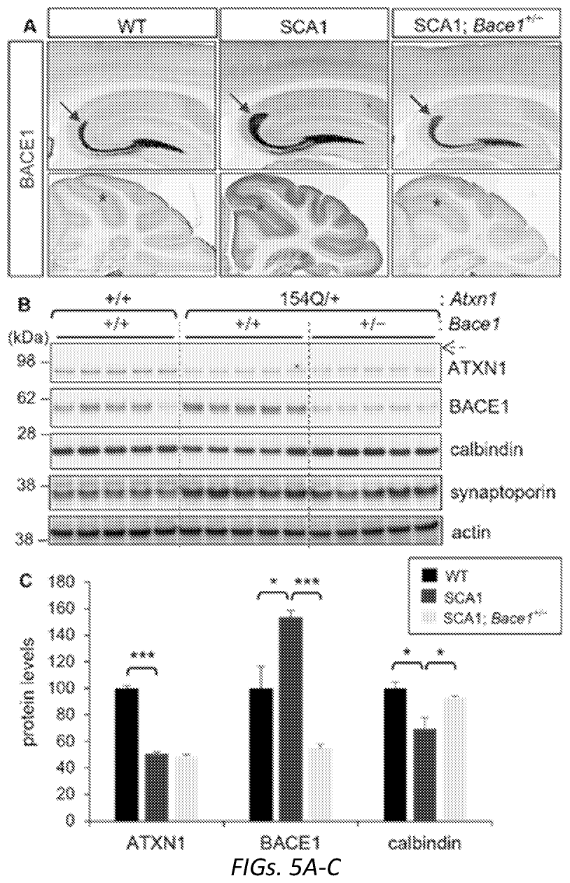

We next examined the effects of BACE1 genetic reduction on neuropathology. Immunohistochemical analysis of 9-month-old Atxn1^{154Q/+} mice revealed BACE1 expression was increased throughout the brain, with ˜35% increase in cortex and ˜50% in cerebellum (FIGS. 5A-B). In the cerebellum, BACE1 increase was most pronounced in the molecular layer where cerebellar granular neurons' parallel axons make extensive amounts of synapses with Purkinje cells' dendrites (FIG. 5A). Similar to BACE1, the expression of synaptoporin, another presynaptic protein, was also markedly increased (69%, p<0.001) in the cerebellum (FIG. 5B) as in hippocampal mossy fibers (Suh et al., 2019).

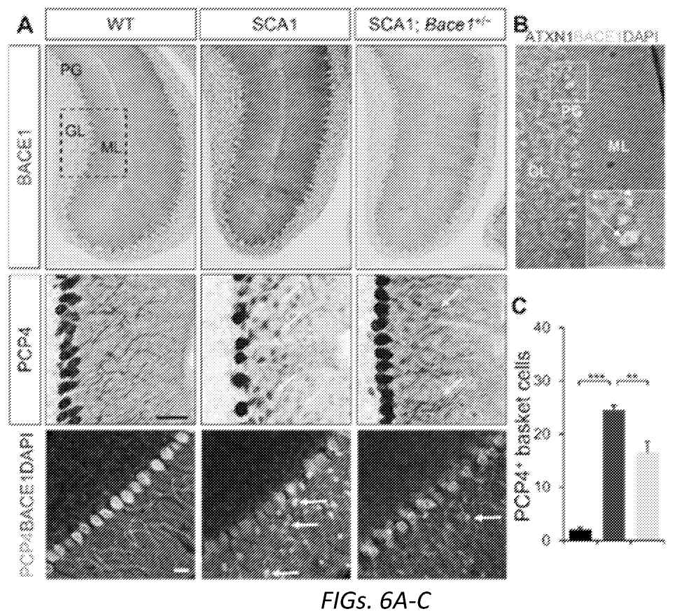

Interestingly, we found Purkinje neurons in cerebellum express high level of BACE1 in the soma, in contrast to the diffused expression pattern of most neurons in the cerebrum (FIG. 6A, 6B). Concentrated BACE1 expression in neuronal soma was also detected in nuclei of brain stem (e.g., hypoglossal and facial neurons) (not shown). These findings suggests that, in the cerebellum and brain stem, two most vulnerable brain regions in SCA, BACE1 can exert its effects directly to neuronal cell body (e.g., excitability of Purkinje neurons) in addition to those mediated through neuronal processes and synapses. Compared to WT, the expressions of calbindin and PCP4 (Purkinje cell protein 4) were significantly decreased in the cerebellum of Atxn1^{154Q/+} mice (FIG. 5B, 6A). However, in Atxn1^{154Q/+}; Bace1^{+/-} littermates, the decreases of Purkinje neuronal markers were attenuated, suggesting BACE1 genetic reduction has rescued the degeneration of Purkinje neurons that are central in cerebellar neuronal circuitry. Unexpectedly, the basket cells in cerebellar molecular layer showed strong PCP4 immunoreactivity in Atxn1^{154Q/+} mice, which was concordant with decreased PCP4 immunoreactivity in nearby Purkinje cells (FIG. 6A, 6C). The PCP4 expression in basket cells was significantly attenuated in Atxn1^{154Q/+} Bace1^{+/-} mice.

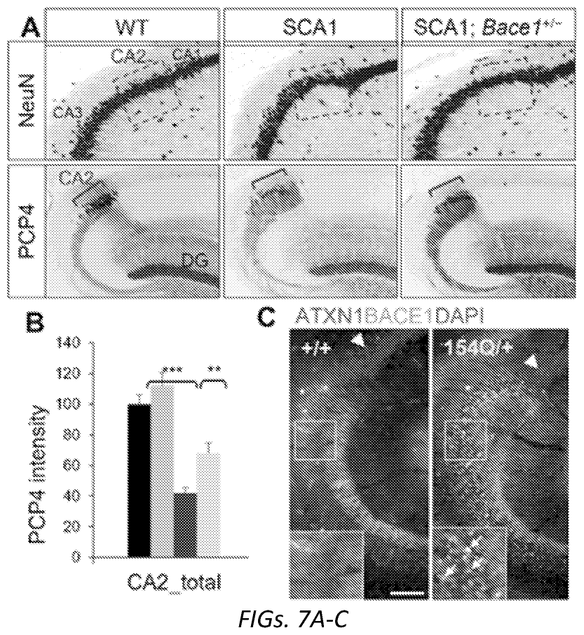

Another brain region revealing distinct BACE1 increase was hippocampal mossy fiber ends near CA2. In addition to increased BACE1 immunoreactivity, the area was significantly enlarged in SCA1 mice (189% of WT) at 9 months old (FIG. 7A, 7C). Notably, in Atxn1^{154Q/+}; Bace1^{+/-} mice, the increases were markedly attenuated in intensity and area size. Immunohistochemical analysis of the hippocampal area with NeuN and PCP4 antibodies revealed that CA2 neurons were selectively degenerated in Atxn1^{154Q/+}, which was significantly attenuated in Atxn1^{154Q/+}; Bace1^{+/-} mice (FIGS. 7A-C).

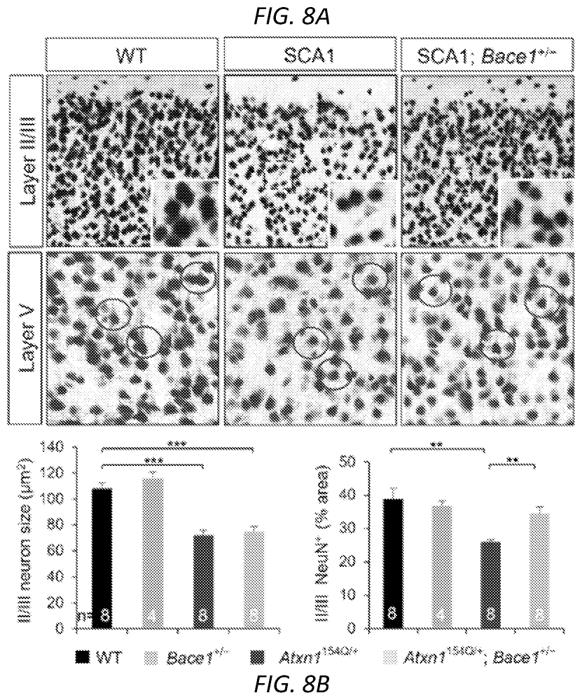

Among other brain regions of the SCA1 mice, we found cortex is one of the most shrunken areas compared to WT control (10% decrease by area and 18% decrease by weight, p<0.001 for each). When motor cortex was examined by neuronal marker NeuN, the size of neuron in cortical layer II/III was significantly smaller in SCA1 than WT mice (FIGS. 8A-B). While the neuronal size was not changed in Atxn1^{154Q/+}; Bace1^{+/-} mice, they have much higher neuronal density than Atxn1^{154Q/+} littermates. In layer V, in contrast to the pyramidal shape of WT neurons, NeuN-positive neurons of SCA1 mice exhibit more rounded cell shape and diminished proximal dendrite. These morphological changes of cortical neurons were significantly rescued in Atxn1^{154Q/+}; Bace1^{+/-} mice.

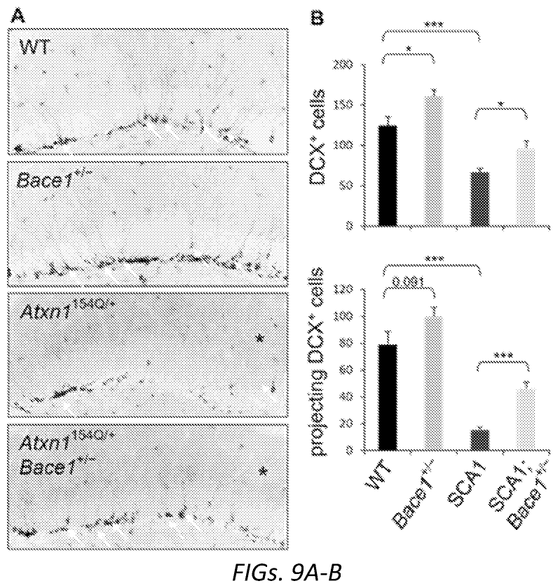

Next, we assessed if BACE1 genetic reduction would affect impaired hippocampal neurogenesis in SCA1 mice (Suh et al., 2019). When analyzed for DCX⁺ newborn neurons, 5-month-old Atxn1^{154Q/+}; Bace1^{+/-} mice showed DCX⁺ neuron number was significant increased and their dendrites were more developed in the subgranular cell layer than those of Atxn1^{154Q/+} littermate (FIGS. 9A-B).

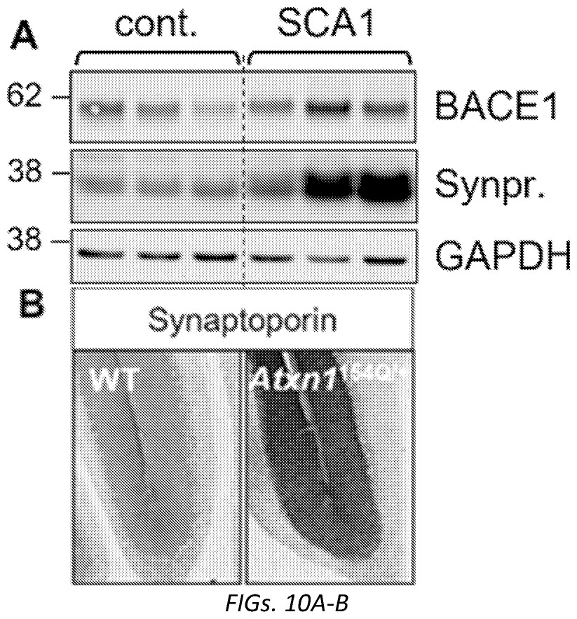

Preliminary analysis for 6 SCA1 and 5 healthy control human brain tissues by Western blot showed that there was a slight trend of BACE1 increase in SCA1 brains in both cerebellum (31%) and cortex (36%), although they were not statistically significant due to large variation among the tissue samples (FIG. 10A). Notably, the expression of synaptoporin, another presynaptic protein, was markedly increased (106%, p=0.05) in concordance with BACE1 levels in SCA1 cerebellar tissues. Synaptoporin expression was greatly increased in the cerebellar molecular layer of SCA1 mice as compared with wild type (FIG. 10B).

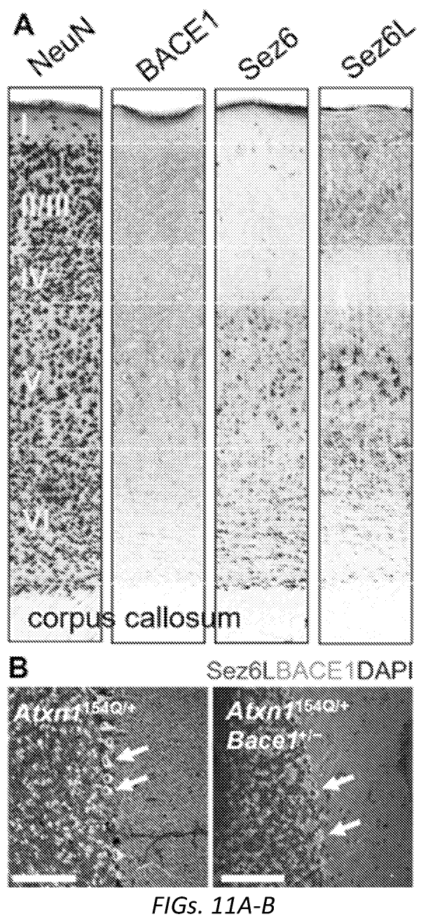

In the cortex, Sez6L is widely expressed with highest levels found in layer 2/3 and 5 neurons and processes (Nash et al., Cerebral cortex. 2020). Here, BACE1 was expressed throughout the cortex and co-expressed with Sez6 and Sez6L in soma of superficial layer 5 pyramidal neurons in WT mice (FIG. 11A); this was confirmed with Bace1 null mice. Furthermore, in the cerebellum Sez6L colocalized with BACE1 in the soma of Purkinje cells (FIG. 11B).

We hypothesized that the rescuing effects of genetic BACE1 reduction are produced through reduced BACE1 cleavage of substrates that play important physiological functions in the brain. To test this hypothesis, we first examined whether the cleavages of known BACE1 substrates are changed in Atxn1^{154Q/+} mice. Beyond serving as the precursor of Ab, APP plays important functions in synaptic plasticity and neuromuscular junctions. In Atxn1^{154Q/+}; Bace1^{+/-} mice, APP cleavage by BACE1 was significantly decreased (42%, p=0.018, data not shown) than that of Atxn1^{154Q/+} mice.

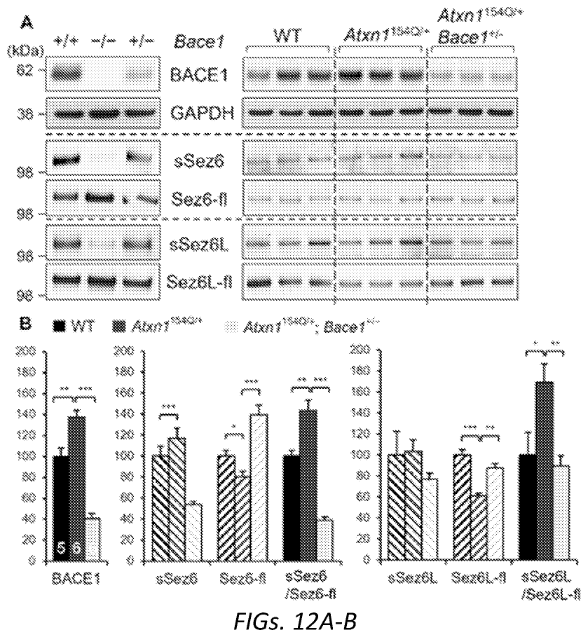

Among other BACE1 substrates, several of recent studies in mice showed that Seizure-related gene 6 (Sez6) and Sez6-Like (Sez6L) proteins play critical roles in cognitive functions and motor coordination (Gunnersen et al., 2007; Nash et al., 2020; Nash et al., 2021; Ong-Palsson et al., 2022). In BACE1 KO mouse brain, BACE1-cleaved secreted Sez6 and Sez6L (sSez6 and sSez6L) were barely detected, whereas membrane-bound uncleaved Sez6 and Sez6L (Sez6-fl and Sez6L-fl) were significantly increased (FIG. 12A), confirming that Sez6 and Sez6L are mainly cleaved by BACE1 in the brain. In the cortex of Atxn1^{154Q/+} mice, where BACE1 level is upregulated ˜35%, changes in Sez6 and Sez6L processing were modest. Nevertheless, the ratios of cleaved ectodomain vs. membrane-bound uncleaved Sez6 and Sez6L were significantly increased (47, 72% respectively) in the SCA1 mice (FIG. 12B), suggesting BACE1 enzymatic activity is increased in the SCA1 mouse brain. In Atxn1^{154Q/+}; Bace1^{+/-} mice, the increases were reversed and returned to the levels similar to or lower than those of WT mice.

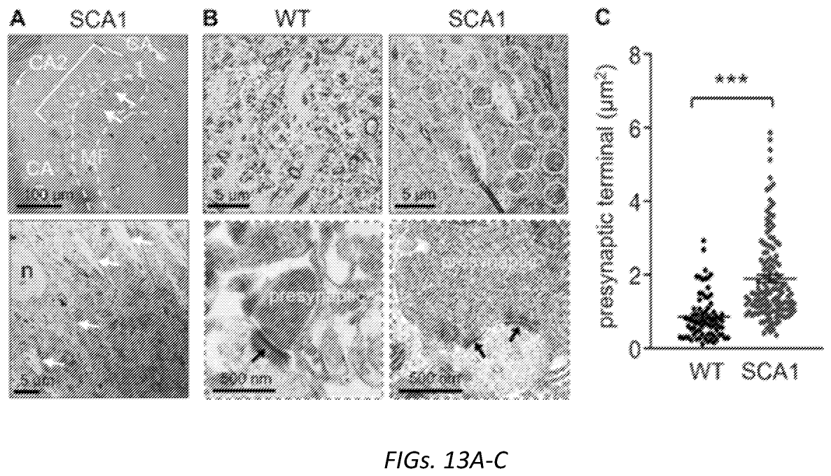

It was further hypothesized that additional factors (e.g., cellular changes) in SCA1 brain affect BACE1 activity, making it more critical in the disease pathogenesis. To test this hypothesis, we first examined the ultrastructure of hippocampal mossy fiber terminal region near CA2, which becomes enlarged as disease progresses and expresses BACE1 highly (FIGS. 5A-C, 7A-C). In the examination of 9-month-old Atxn1^{154Q/+} mice with transmission electron microscopy (EM), we found the proximal dendrites of CA2 neurons are markedly larger than those of WT (FIG. 13A). Among other subcellular structures, the size of presynaptic terminals filled with synaptic vesicles was notably increased (˜2-fold) in the SCA1 mice (FIG. 13B,C).

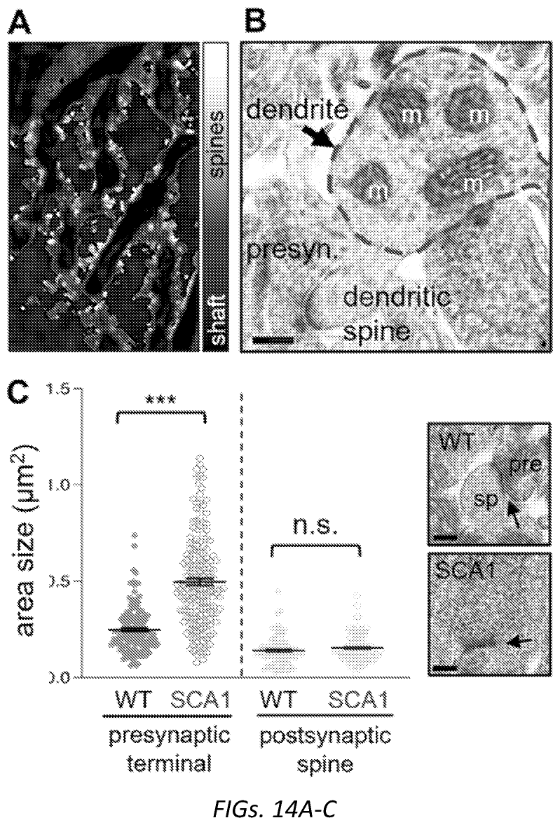

The molecular layer of cerebellum is another region where BACE1 expression is high because parallel axons of cerebellar granular neurons make large amounts of synapses with Purkinje cell's dendrites (˜100,000/PC). Concordant with the findings in hippocampus, presynaptic terminals in the cerebellar region were significantly larger than those of WT while the size of postsynaptic spine head is barely changed (FIGS. 14A-C). These findings reveal that, in addition to increased BACE1 expression, synaptic structures where BACE1 is highly expressed were changed in SCA1 brains.

### Example 6. Effect of Chronic Pharmacological Inhibition of BACE1 on SCA1 Pathology and Behavioral Deficits

While in a few studies BACE1 was shown to produce its effects through direct protein-protein interactions (e.g., ion channel formation) (Hessler et al, 2015; Huth et al, 2009; Lehnert et al, 2016), most of BACE1's cellular functions are believed via its proteolytic activity on substrates. We test whether chronic inhibition of BACE1 protease activity would produce beneficial effects on SCA1 phenotypes, similar to those observed after BACE1 genetic reduction in the preliminary study. We utilize MBi-9, developed by Merck, that has similar structure and properties of BACE1 inhibitor verubecestat (MK-8931) tested in multiple animal models and phase III AD clinical trials (Egan et al., 2018; Egan et al, 2019a; Kennedy et al., 2016). We test the efficacy of BACE1 inhibitor in a wide range of concentration, from very low (<1 mg/kg) to high concentration (30 mg/kg). This is because the Bace1^{+/-} conditions that produced beneficial effects on SCA1 mice represent a partial BACE1 inhibition that is likely equivalent to low dose of BACE1 inhibitor (a few mg/kg or lower concentration). In contrast, in most of the previous preclinical or clinical studies BACE1 inhibitors were used in relatively high concentrations (e.g., 30 mg/kg) and produced near complete BACE1 inhibition, similar to those observed in BACE1 KO background (Kennedy et al., 2016; McDade et al., 2021), on its cleavage of its substrates, such as APP (Kuhn et al, 2012), Sez6/Sez6L (Nash et al., 2021) and Nrg1 (Cheret et al., 2013).

BACE1 inhibitor: MBi-9, described in this example, is provided from Merck & Co. MBi-9 is orally bioavailable, potent and selective BACE1 inhibitor (Ki=˜2 nM; ˜100,000-fold selective over non-BACE proteases; see Table 1) that has been shown to effectively reduce CNS AB40 levels by >90% in rodents at 30 mg/kg/day dose. To achieve sustained and long-term suppression of BACE activity, MBi-9 is custom-formulated in chow with different doses (Research Diets, New Brunswick, NJ) to deliver different level of chronic BACE1 inhibition (Tallon et al, 2020). In preclinical and clinical studies, MBi-9, MBi-5, or MK-8931, three related BACE1 inhibitors, did not produce any serious adverse side effects such as liver toxicity (Dobrowolska et al, 2014; Kennedy et al., 2016; Tallon et al., 2020). Prior study with verubecestat and MBi-9 treatment showed that full target engagement and BACE1 inhibition was achieved within 24 hr after the drug administration.

TABLE 1
MBi-9 profile

| ENZYME OR CELL LINE | MBi-9 Kᵢ or IC₅₀ (nM) |
|---|---|
| BACE1 | 1.5 ± 0.7 |
| Mouse BACE1 | 2.3 ± 0.9 |
| BACE2 | 2.3 ± 1.4 |
| Mouse BACE2 | 5.0 ± 1.9 |
| CATHEPSIN D | 37380 ± 9000 |
| CATHEPSIN E | >100,000 |
| PEPSIN | >100,000 |
| RENIN | 16402 ± 1772 |
| HEK293 APP^{SWE/LON}Aβ₄₀ IC₅₀ | 2.2 ± 2.0 |
| HEK293 APP^{SWE/LON}Aβ₄₂ IC₅₀ | 2.6 ± 2.1 |
| HEK293 APP^{SWE/LON}SAPPβ IC₅₀ | 16 |

Other BACE1 inhibitors, e.g., as described herein, may also be used in these experiments.

Pharmacokinetic and pharmacodynamic (PK/PD) analysis: The in-diet CNS PK profile is examined for Atxn1^{154Q/+} SCA1 mice prior to conducting efficacy study (Kennedy et al., 2016; Scott et al, 2016; Tallon et al., 2020). Four-month-old SCA1 and WT mice are split into 6 different groups and given for 10 days either 0, 0.3, 1, 3, 10, or 30 mg/kg doses of MBi-9 chow (n=4/group). The amount of food eaten per day and the daily body weight are measured throughout the dosing period. After 10-day treatment, mice are euthanized and blood and brain tissues are collected. The actual doses are calculated by multiplying the dose in the chow by the amount of chow divided by mouse body weight (diet dose×chow eaten/body weight). The amount of drug in the blood plasma and brain samples are determined by liquid chromatography coupled with tandem mass spectrometry as described previously (Kennedy et al., 2016; Mandal et al, 2016).

To examine dose-dependent effects of MBi-9 on its substrates, we analyze changes in the cleavage of well-known BACE1 substrates, such as APP and Sez6/Sez6L. Western blot analysis of membrane and soluble fraction of MBi-9-treated mouse brains is performed to determine levels of BACE1 inhibition, as compared to those observed in Bace1^{+/-} or Bace1^{+/-} background.

Chronic drug treatment and behavioral and survival tests: Atxn1^{154Q/+} mice will be given the chow containing MBi-9 from 6 weeks old of age to the end of study. The SCA1 mice exhibited some deficits at 6 weeks in rotarod test (Watase et al, 2002) and slight BACE1 increase was observed in hippocampus (Suh et al., 2019). However, we avoid BACE1 inhibitor treatment for too young mice (>1 month) as it could cause unexpected developmental problem related to BACE1 inhibition (e.g., hypomyelination) (Hu et al, 2006; Willem et al, 2006). MBi-9 treatment is split into different dose groups, e.g., 0, 0.3, 1, 3, 10, 30 mg/kg/day. Twenty mice per each dose group will be tested for behaviors (rotarod, open field, and beam walking) and then examined for lifespan change, as described above.

Neurodegeneration and electrophysiology analysis: Once the most effective doses are determined from the behavioral and survival tests, those doses are used to treat and examine whether MBi-9 would attenuate SCA1 pathology. A cohort of mice per dose group is treated with the effective MBi-9 doses for 8 months. Neurodegeneration in motor cortex, hippocampus, and cerebellum is examined as described above. Remaining mice per each group are utilized for electrophysiological recordings of Purkinje neurons as described above.

### REFERENCES

Ashizawa, T., Oz, G., and Paulson, H. L. (2018). Spinocerebellar ataxias: prospects and challenges for therapy development. Nat Rev Neurol 14, 590-605. 10.1038/s41582-018-0051-6.
Barao, S., Gartner, A., Leyva-Diaz, E., Demyanenko, G., Munck, S., Vanhoutvin, T., Zhou, L., Schachner, M., Lopez-Bendito, G., Maness, P. F., and De Strooper, B. (2015). Antagonistic Effects of BACE1 and APH1B-gamma-Secretase Control Axonal Guidance by Regulating Growth Cone Collapse. Cell Rep 12, 1367-1376. S2211-1247 (15) 00849-9 [pii]
Baroy T, Misceo D, Stromme P, Stray-Pedersen A, Holmgren A, Rodningen O K, Blomhoff A, Helle J R, Stormyr A, Tvedt B et al (2013) Haploinsufficiency of two histone modifier genes on 6p22.3, ATXN1 and JARID2, is associated with intellectual disability. Orphanet J Rare Dis 8:3
Bertram L, Lange C, Mullin K, Parkinson M, Hsiao M, Hogan M F, Schjeide B M, Hooli B, Divito J, Ionita I et al (2008) Genome-wide association analysis reveals putative Alzheimer's disease susceptibility loci in addition to APOE. Am J Hum Genet 83:623-632
Bettens K, Brouwers N, Van Miegroet H, Gil A, Engelborghs S, De Deyn P P, Vandenberghe R, Van Broeckhoven C, Sleegers K (2010) Follow-up study of susceptibility loci for Alzheimer's disease and onset age identified by genome-wide association. J Alzheimers Dis 19:1169-1175
Brooker, S. M., Edamakanti, C. R., Akasha, S. M., Kuo, S. H., and Opal, P. (2021). Spinocerebellar ataxia clinical trials: opportunities and challenges. Ann Clin Transl Neurol 8, 1543-1556. 10.1002/acn3.51370.
Bushart D D, Huang H, Man L J, Morrison L M, Shakkottai V G (2021) A Chlorzoxazone-Baclofen Combination Improves Cerebellar Impairment in Spinocerebellar Ataxia Type 1. Mov Disord 36:622-631
Celestino-Soper P B, Skinner C, Schroer R, Eng P, Shenai J, Nowaczyk M M, Terespolsky D, Cushing D, Patel G S, Immken L et al (2012) Deletions in chromosome 6p22.3-p24.3, including ATXN1, are associated with developmental delay and autism spectrum disorders. Mol Cytogenet 5:17
Cheret C, Willem M, Fricker F R, Wende H, Wulf-Goldenberg A, Tahirovic S, Nave K A, Saftig P, Haass C, Garratt A N et al (2013) Bace1 and Neuregulin-1 cooperate to control formation and maintenance of muscle spindles. EMBO J 32:2015-2028
Chopra R, Wasserman A H, Pulst S M, De Zeeuw C I, Shakkottai V G (2018) Protein kinase C activity is a protective modifier of Purkinje neuron degeneration in cerebellar ataxia. Hum Mol Genet 27:1396-1410
Coarelli, G., Heinzmann, A., Ewenczyk, C., Fischer, C., Chupin, M., Monin, M. L., Hurmic, H., Calvas, F., Calvas, P., Goizet, C., et al. (2022). Safety and efficacy of riluzole in spinocerebellar ataxia type 2 in France (ATRIL): a multicentre, randomised, double-blind, placebo-controlled trial. Lancet Neurol 21, 225-233. 10.1016/S1474-4422 (21) 00457-9.
Das B, Yan R (2019) A Close Look at BACE1 Inhibitors for Alzheimer's Disease Treatment. CNS Drugs 33:251-263
Di Benedetto D, Di Vita G, Romano C, Giudice M L, Vitello G A, Zingale M, Grillo L, Castiglia L, Musumeci S A, Fichera M (2013) 6p22.3 deletion: report of a patient with autism, severe intellectual disability and electroencephalographic anomalies. Mol Cytogenet 6:4
Dobrowolska J A, Michener M S, Wu G, Patterson B W, Chott R, Ovod V, Pyatkivskyy Y, Wildsmith K R, Kasten T, Mathers P et al (2014) CNS amyloid-beta, soluble APP-alpha and -beta kinetics during BACE inhibition. J Neurosci 34:8336-8346
Egan M F, Kost J, Tariot P N, Aisen P S, Cummings J L, Vellas B, Sur C, Mukai Y, Voss T, Furtek C et al (2018) Randomized Trial of Verubecestat for Mild-to-Moderate Alzheimer's Disease. N Engl J Med 378:1691-1703
Egan M F, Kost J, Voss T, Mukai Y, Aisen P S, Cummings J L, Tariot P N, Vellas B, van Dyck C H, Boada M et al (2019a) Randomized Trial of Verubecestat for Prodromal Alzheimer's Disease. N Engl J Med 380:1408-1420
Egan M F, Mukai Y, Voss T, Kost J, Stone J, Furtek C, Mahoney E, Cummings J L, Tariot P N, Aisen P S et al (2019b) Further analyses of the safety of verubecestat in the phase 3 EPOCH trial of mild-to-moderate Alzheimer's disease. Alzheimers Res Ther 11:68
Farah, M. H., Pan, B. H., Hoffman, P. N., Ferraris, D., Tsukamoto, T., Nguyen, T., Wong, P. C., Price, D. L., Slusher, B. S., and Griffin, J. W. (2011). Reduced BACE1 activity enhances clearance of myelin debris and regeneration of axons in the injured peripheral nervous system. J Neurosci 31, 5744-5754. 31/15/5744 [pii]
Finkel, R. S., Mercuri, E., Darras, B. T., Connolly, A. M., Kuntz, N. L., Kirschner, J., Chiriboga, C. A., Saito, K., Servais, L., Tizzano, E., et al. (2017). Nusinersen versus Sham Control in Infantile-Onset Spinal Muscular Atrophy. N Engl J Med 377, 1723-1732. 10.1056/NEJMoa1702752.
Friedrich, J., Kordasiewicz, H. B., O'Callaghan, B., Handler, H. P., Wagener, C., Duvick, L., Swayze, E. E., Rainwater, O., Hofstra, B., Benneyworth, M., et al. (2018). Antisense oligonucleotide-mediated ataxin-1 reduction prolongs survival in SCA1 mice and reveals disease-associated transcriptome profiles. JCI Insight 3. 10.1172/jci.insight. 123193.
Fryer, J. D., Yu, P., Kang, H., Mandel-Brehm, C., Carter, A. N., Crespo-Barreto, J., Gao, Y., Flora, A., Shaw, C., Orr, H. T., and Zoghbi, H. Y. (2011). Exercise and genetic rescue of SCA1 via the transcriptional repressor Capicua. Science 334, 690-693. 334/6056/690 [pii]
Gunnersen J M, Kim M H, Fuller S J, De Silva M, Britto J M, Hammond V E, Davies P J, Petrou S, Faber E S, Sah P et al (2007) Sez-6 proteins affect dendritic arborization patterns and excitability of cortical pyramidal neurons. Neuron 56:621-639
Gusella, J. F. (2019). CAG Repeat Not Polyglutamine Length Determines Timing of Huntington's Disease Onset. Cell 178, 887-900 e814. S0092-8674 (19) 30739-1 [pii]
Guyenet S J, Furrer S A, Damian V M, Baughan T D, La Spada A R, Garden G A (2010) A simple composite phenotype scoring system for evaluating mouse models of cerebellar ataxia. J Vis Exp (39): 1787
Hampel H, Vassar R, De Strooper B, Hardy J, Willem M, Singh N, Zhou J, Yan R, Vanmechelen E, De Vos A et al (2021) The beta-Secretase BACE1 in Alzheimer's Disease. Biol Psychiatry 89:745-756
Hampel, H., Vassar, R., De Strooper, B., Hardy, J., Willem, M., Singh, N., Zhou, J., Yan, R., Vanmechelen, E., De Vos, A., et al. (2021). The beta-Secretase BACE1 in Alzheimer's Disease. Biol Psychiatry 89, 745-756. 10.1016/j.biopsych.2020.02.001.
Hessler S, Zheng F, Hartmann S, Rittger A, Lehnert S, Volkel M, Nissen M, Edelmann E, Saftig P, Schwake M et al (2015) beta-Secretase BACE1 regulates hippocampal and reconstituted M-currents in a beta-subunit-like fashion. J Neurosci 35:3298-3311
Hinkle, C. L., Diestel, S., Lieberman, J., and Maness, P. F. (2006). Metalloprotease-induced ectodomain shedding of neural cell adhesion molecule (NCAM). J Neurobiol 66, 1378-1395. 10.1002/neu.20257.
Hourez R, Servais L, Orduz D, Gall D, Millard I, de Kerchove d'Exaerde A, Cheron G, Orr H T, Pandolfo M, Schiffmann S N (2011) Aminopyridines correct early dysfunction and delay neurodegeneration in a mouse model of spinocerebellar ataxia type 1. J Neurosci 31:11795-11807
Hu X, Hicks C W, He W, Wong P, Macklin W B, Trapp B D, Yan R (2006) Bace1 modulates myelination in the central and peripheral nervous system. Nat Neurosci 9:1520-1525
Huth T, Schmidt-Neuenfeldt K, Rittger A, Saftig P, Reiss K, Alzheimer C (2009) Non-proteolytic effect of beta-site APP-cleaving enzyme 1 (BACE1) on sodium channel function. Neurobiol Dis 33:282-289
Jia, D. D., Zhang, L., Chen, Z., Wang, C. R., Huang, F. Z., Duan, R. H., Xia, K., Tang, B. S., and Jiang, H. (2013). Lithium chloride alleviates neurodegeneration partly by inhibiting activity of GSK3beta in a SCA3 Drosophila model. Cerebellum 12, 892-901. 10.1007/s12311-013-0498-3.
Kennedy M E, Stamford A W, Chen X, Cox K, Cumming J N, Dockendorf M F, Egan M, Ereshefsky L, Hodgson R A, Hyde L A et al (2016) The BACE1 inhibitor verubecestat (MK-8931) reduces CNS beta-amyloid in animal models and in Alzheimer's disease patients. Sci Transl Med 8: 363ra150
Kim, D. Y., Carey, B. W., Wang, H., Ingano, L. A., Binshtok, A. M., Wertz, M. H., Pettingell, W. H., He, P., Lee, V. M., Woolf, C. J., and Kovacs, D. M. (2007). BACE1 regulates voltage-gated sodium channels and neuronal activity. Nat Cell Biol 9, 755-764. ncb 1602 [pii]
Kim, J., Hu, C., Moufawad El Achkar, C., Black, L. E., Douville, J., Larson, A., Pendergast, M. K., Goldkind, S. F., Lee, E. A., Kuniholm, A., et al. (2019). Patient-Customized Oligonucleotide Therapy for a Rare Genetic Disease. N Engl J Med 381, 1644-1652. 10.1056/NEJMoa1813279.
Kingwell, K. (2021). Double setback for ASO trials in Huntington disease. Nat Rev Drug Discov 20, 412-413. 10.1038/d41573-021-00088-6.
Klockgether T, Mariotti C, Paulson H L (2019) Spinocerebellar ataxia. Nat Rev Dis Primers 5:24
Klockgether, T., Mariotti, C., and Paulson, H. L. (2019). Spinocerebellar ataxia. Nat Rev Dis Primers 5, 24. 10.1038/s41572-019-0074-3.
Kuhn P H, Koroniak K, Hogl S, Colombo A, Zeitschel U, Willem M, Volbracht C, Schepers U, Imhof A, Hoffmeister A et al (2012) Secretome protein enrichment identifies physiological BACE1 protease substrates in neurons. EMBO J 31:3157-3168
Kwon, D. (2021). Failure of genetic therapies for Huntington's devastates community. Nature 593, 180. 10.1038/d41586-021-01177-7.
Laird F M, Cai H, Savonenko A V, Farah M H, He K, Melnikova T, Wen H, Chiang H C, Xu G, Koliatsos V E et al (2005) BACE1, a major determinant of selective vulnerability of the brain to amyloid-beta amyloidogenesis, is essential for cognitive, emotional, and synaptic functions. J Neurosci 25:11693-11709
Lehnert S, Hartmann S, Hessler S, Adelsberger H, Huth T, Alzheimer C (2016) Ion channel regulation by beta-secretase BACE1-enzymatic and non-enzymatic effects beyond Alzheimer's disease. Channels (Austin) 10:365-378
Lei, L. F., Yang, G. P., Wang, J. L., Chuang, D. M., Song, W. H., Tang, B. S., and Jiang, H. (2016). Safety and efficacy of valproic acid treatment in SCA3/MJD patients. Parkinsonism Relat Disord 26, 55-61. 10.1016/j.parkreldis.2016.03.005.
Loane, D. J., Pocivavsek, A., Moussa, C. E., Thompson, R., Matsuoka, Y., Faden, A. I., Rebeck, G. W., and Burns, M. P. (2009). Amyloid precursor protein secretases as therapeutic targets for traumatic brain injury. Nat Med 15, 377-379. nm. 1940 [pii]
Lomoio, S., Willen, R., Kim, W., Ho, K. Z., Robinson, E. K., Prokopenko, D., Kennedy, M. E., Tanzi, R. E., and Tesco, G. (2020). Gga3 deletion and a GGA3 rare variant associated with late onset Alzheimer's disease trigger BACE1 accumulation in axonal swellings. Sci Transl Med 12. 10.1126/scitranslmed.aba1871.
Lu H C, Tan Q, Rousseaux M W, Wang W, Kim J Y, Richman R, Wan Y W, Yeh S Y, Patel J M, Liu X et al (2017) Disruption of the ATXN1-CIC complex causes a spectrum of neurobehavioral phenotypes in mice and humans. Nat Genet 49:527-Luong, T. N., Carlisle, H. J., Southwell, A., and Patterson, P. H. (2011). Assessment of motor balance and coordination in mice using the balance beam. J Vis Exp. 10.3791/2376.
Mandal M, Wu Y, Misiaszek J, Li G, Buevich A, Caldwell J P, Liu X, Mazzola R D, Orth P, Strickland C et al (2016) Structure-Based Design of an Iminoheterocyclic beta-Site Amyloid Precursor Protein Cleaving Enzyme (BACE) Inhibitor that Lowers Central Abeta in Nonhuman Primates. J Med Chem 59:3231-3248
McDade E, Voytyuk I, Aisen P, Bateman R J, Carrillo M C, De Strooper B, Haass C, Reiman E M, Sperling R, Tariot P N et al (2021) The case for low-level BACE1 inhibition for the prevention of Alzheimer disease. Nat Rev Neurol 17:703-714
McLoughlin H S, Moore L R, Chopra R, Komlo R, Mckenzie M, Blumenstein K G, Zhao H, Kordasiewicz H B, Shakkottai V G, Paulson H L (2018) Oligonucleotide therapy mitigates disease in spinocerebellar ataxia type 3 mice. Ann Neurol 84:64-77
Mercuri, E., Darras, B. T., Chiriboga, C. A., Day, J. W., Campbell, C., Connolly, A. M., Iannaccone, S. T., Kirschner, J., Kuntz, N. L., Saito, K., et al. (2018). Nusinersen versus Sham Control in Later-Onset Spinal Muscular Atrophy. N Engl J Med 378, 625-635. 10.1056/NEJMoa1710504.
Mundwiler A, Shakkottai V G (2018) Autosomal-dominant cerebellar ataxias. Handb Clin Neurol 147:173-185
Nash A, Aumann T D, Pigoni M, Lichtenthaler S F, Takeshima H, Munro K M, Gunnersen J M (2020) Lack of Sez6 Family Proteins Impairs Motor Functions, Short-Term Memory, and Cognitive Flexibility and Alters Dendritic Spine Properties. Cereb Cortex 30:2167-2184
Nash A, Gijsen H J M, Hrupka B J, Teng K S, Lichtenthaler S F, Takeshima H, Gunnersen J M, Munro K M (2021) BACE inhibitor treatment of mice induces hyperactivity in a Seizure-related gene 6 family dependent manner without altering learning and memory. Sci Rep 11:15084
Ong-Palsson E, Njavro J R, Wilson Y, Pigoni M, Schmidt A, Muller S A, Meyer M, Hartmann J, Busche M A, Gunnersen J M et al (2022) The beta-Secretase Substrate Seizure 6-Like Protein (SEZ6L) Controls Motor Functions in Mice. Mol Neurobiol 59:1183-1198
Orr H T, Chung M Y, Banfi S, Kwiatkowski T J, Jr., Servadio A, Beaudet A L, McCall A E, Duvick L A, Ranum L P, Zoghbi H Y (1993) Expansion of an unstable trinucleotide CAG repeat in spinocerebellar ataxia type 1. Nat Genet 4:221-226
Ou-Yang, M. H., Kurz, J. E., Nomura, T., Popovic, J., Rajapaksha, T. W., Dong, H., Contractor, A., Chetkovich, D. M., Tourtellotte, W. G., and Vassar, R. (2018). Axonal organization defects in the hippocampus of adult conditional BACE1 knockout mice. Sci Transl Med 10. 10/459/eaao5620 [pii]
Paulson H L, Shakkottai V G, Clark H B, Orr H T (2017) Polyglutamine spinocerebellar ataxias—from genes to potential treatments. Nat Rev Neurosci 18: 613-626
Paulson, H. (2018). Repeat expansion diseases. Handb Clin Neurol 147, 105-123. 10.1016/B978-0-444-63233-3.00009-9.
Rice, H. C., de Malmazet, D., Schreurs, A., Frere, S., Van Molle, I., Volkov, A. N., Creemers, E., Vertkin, I., Nys, J., Ranaivoson, F. M., et al. (2019). Secreted amyloid-beta precursor protein functions as a GABABR1a ligand to modulate synaptic transmission. Science 363. 363/6423/eaao4827 [pii]
Romano, S., Coarelli, G., Marcotulli, C., Leonardi, L., Piccolo, F., Spadaro, M., Frontali, M., Ferraldeschi, M., Vulpiani, M. C., Ponzelli, F., et al. (2015). Riluzole in patients with hereditary cerebellar ataxia: a randomised, double-blind, placebo-controlled trial. Lancet Neurol 14, 985-991. 10.1016/S1474-4422 (15) 00201-X.
Rousseaux M W C, Tschumperlin T, Lu H C, Lackey E P, Bondar V V, Wan Y W, Tan Q, Adamski C J, Friedrich J, Twaroski K et al (2018) ATXN1-CIC Complex Is the Primary Driver of Cerebellar Pathology in Spinocerebellar Ataxia Type 1 through a Gain-of-Function Mechanism. Neuron 97:1235-1243 e1235
Sadleir, K. R., Kandalepas, P. C., Buggia-Prevot, V., Nicholson, D. A., Thinakaran, G., and Vassar, R. Presynaptic dystrophic neurites surrounding amyloid plaques are sites of microtubule disruption, BACE1 elevation, and increased Abeta generation in Alzheimer's disease. Acta Neuropathol 132, 235-256. 10.1007/s00401-016-1558-9
Satir, T. M., Agholme, L., Karlsson, A., Karlsson, M., Karila, P., Illes, S., Bergstrom, P., and Zetterberg, H. (2020). Partial reduction of amyloid beta production by beta-secretase inhibitors does not decrease synaptic transmission. Alzheimers Res Ther 12, 63. 10.1186/s13195-020-00635-0.
Scoles D R, Meera P, Schneider M D, Paul S, Dansithong W, Figueroa K P, Hung G, Rigo F, Bennett C F, Otis T S et al (2017) Antisense oligonucleotide therapy for spinocerebellar ataxia type 2. Nature 544:362-366
Scott J D, Li S W, Brunskill A P, Chen X, Cox K, Cumming J N, Forman M, Gilbert E J, Hodgson R A, Hyde L A et al (2016) Discovery of the 3-Imino-1,2,4-thiadiazinane 1,1-Dioxide Derivative Verubecestat (MK-8931)-A beta-Site Amyloid Precursor Protein Cleaving Enzyme 1 Inhibitor for the Treatment of Alzheimer's Disease. J Med Chem 59:10435-10450
Shakkottai V G, do Carmo Costa M, Dell'Orco J M, Sankaranarayanan A, Wulff H, Paulson H L (2011) Early changes in cerebellar physiology accompany motor dysfunction in the polyglutamine disease spinocerebellar ataxia type 3. J Neurosci 31:13002-13014
Suh J, Romano D M, Nitschke L, Herrick S P, DiMarzio B A, Dzhala V, Bae J S, Oram M K, Zheng Y, Hooli B et al (2019) Loss of Ataxin-1 Potentiates Alzheimer's Pathogenesis by Elevating Cerebral BACE1 Transcription. Cell 178:1159-1175 e1117
Sun, X., He, G., Qing, H., Zhou, W., Dobie, F., Cai, F., Staufenbiel, M., Huang, L. E., and Song, W. (2006). Hypoxia facilitates Alzheimer's disease pathogenesis by up-regulating BACE1 gene expression. Proc Natl Acad Sci USA 103, 18727-18732. 0606298103 [pii]
Tallon, C., Marshall, K. L., Kennedy, M. E., Hyde, L. A., and Farah, M. H. (2020). Pharmacological BACE Inhibition Improves Axonal Regeneration in Nerve Injury and Disease Models. Neurotherapeutics. 10.1007/s13311-020-00852-3
Tallon, C., Rockenstein, E., Masliah, E., and Farah, M. H. (2017). Increased BACE1 activity inhibits peripheral nerve regeneration after injury. Neurobiol Dis 106, 147-157. S0969-9961 (17) 30155-9 [pii]
Tejwani L, Lim J (2020) Pathogenic mechanisms underlying spinocerebellar ataxia type 1. Cell Mol Life Sci 77:4015-4029
Tejwani, L., and Lim, J. (2020). Pathogenic mechanisms underlying spinocerebellar ataxia type 1. Cell Mol Life Sci 77, 4015-4029. 10.1007/s00018-020-03520-z.
Tesco, G., Koh, Y. H., Kang, E. L., Cameron, A. N., Das, S., Sena-Esteves, M., Hiltunen, M., Yang, S. H., Zhong, Z., Shen, Y., et al. (2007). Depletion of GGA3 stabilizes BACE and enhances beta-secretase activity. Neuron 54, 721-737. 10.1016/j.neuron.2007.05.012.
Vassar, R., Kuhn, P. H., Haass, C., Kennedy, M. E., Rajendran, L., Wong, P. C., and Lichtenthaler, S. F. (2014). Function, therapeutic potential and cell biology of BACE proteases: current status and future prospects. J Neurochem 130, 4-28. 10.1111/jnc.12715.
Walker, K. R., Kang, E. L., Whalen, M. J., Shen, Y., and Tesco, G. (2012). Depletion of GGA1 and GGA3 mediates postinjury elevation of BACE1. J Neurosci 32, 10423-10437. 10.1523/JNEUROSCI.5491-11.2012.
Watase K, Weeber E J, Xu B, Antalffy B, Yuva-Paylor L, Hashimoto K, Kano M, Atkinson R, Sun Y, Armstrong D L et al (2002) A long CAG repeat in the mouse Sca1 locus replicates SCA1 features and reveals the impact of protein solubility on selective neurodegeneration. Neuron 34:905-919
Watase, K., Gatchel, J. R., Sun, Y., Emamian, E., Atkinson, R., Richman, R., Mizusawa, H., Orr, H. T., Shaw, C., and Zoghbi, H. Y. (2007). Lithium therapy improves neurological function and hippocampal dendritic arborization in a spinocerebellar ataxia type 1 mouse model. PLOS Med 4, e182. 10.1371/journal.pmed.0040182.
Weyer, S. W., Zagrebelsky, M., Herrmann, U., Hick, M., Ganss, L., Gobbert, J., Gruber, M., Altmann, C., Korte, M., Deller, T., and Muller, U. C. (2014). Comparative analysis of single and combined APP/APLP knockouts reveals reduced spine density in APP-KO mice that is prevented by APPsalpha expression. Acta Neuropathol Commun 2, 36. 2051-5960-2-36 [pii]
Willem M, Garratt A N, Novak B, Citron M, Kaufmann S, Rittger A, DeStrooper B, Saftig P, Birchmeier C, Haass C (2006) Control of peripheral nerve myelination by the beta-secretase BACE1. Science 314:664-666
Wong, H. K., Sakurai, T., Oyama, F., Kaneko, K., Wada, K., Miyazaki, H., Kurosawa, M., De Strooper, B., Saftig, P., and Nukina, N. (2005). beta Subunits of voltage-gated sodium channels are novel substrates of beta-site amyloid precursor protein-cleaving enzyme (BACE1) and gamma-secretase. J Biol Chem 280, 23009-23017. M414648200 [pii]
Wright, A. G., Demyanenko, G. P., Powell, A., Schachner, M., Enriquez-Barreto, L., Tran, T. S., Polleux, F., and Maness, P. F. (2007). Close homolog of L1 and neuropilin 1 mediate guidance of thalamocortical axons at the ventral telencephalon. J Neurosci 27, 13667-13679. 27/50/13667 [pii]
Zhang, X., Zhou, K., Wang, R., Cui, J., Lipton, S. A., Liao, F. F., Xu, H., and Zhang, Y. W. (2007). Hypoxia-inducible factor 1alpha (HIF-1alpha)-mediated hypoxia increases BACE1 expression and beta-amyloid generation. J Biol Chem 282, 10873-10880. M608856200 [pii]
Zhao, J., Fu, Y., Yasvoina, M., Shao, P., Hitt, B., O'Connor, T., Logan, S., Maus, E., Citron, M., Berry, R., et al. (2007). Beta-site amyloid precursor protein cleaving enzyme 1 levels become elevated in neurons around amyloid plaques: implications for Alzheimer's disease pathogenesis. J Neurosci 27, 3639-3649. 27/14/3639 [pii].
Zoghbi H Y, Orr H T (2000) Glutamine repeats and neurodegeneration. Annu Rev Neurosci 23:217-247.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of treating a subject who has a neurodegenerative condition associated with loss of motor function, the method comprising administering a therapeutically effective amount of an inhibitor of BACE1.

2. The method of claim 1, wherein the inhibitor of BACE1 is a small molecule inhibitor of BACE1.

3. The method of claim 2, wherein the small molecule inhibitor of BACE1 is selected from the group consisting of verubecestat, MBI-10, MBI-1, MBI-3, MBI-5, MBI-9, LY2886721, LY2811376, LY3202626, elenbecestat, RG7129, TAK-070, CTS-21166, lanabecestat, AZ4217, HPP854, ginsenoside Rg1, BI 1181181, hispidin, TDC (CID 5811533), umibecestat, Monacolin K, PF-05297909, PF-06751979, CTS2116, atabecestat, RG7129 (RO5508887), SCH 1359113, a spirocyclic inhibitor (R)-50), or a fluorine-substituted 1,3-oxazine.

4. The method of claim 1, wherein the inhibitor of BACE1 is an antibody that binds to BACE1, optionally a bispecific antibody or a camelid antibody that binds and inhibits BACE1.

5. The method of claim 1, wherein the inhibitor of BACE1 is an inhibitory oligonucleotide targeting BACE1 that decreases BACE1 expression.

6. The method of claim 5, wherein the oligonucleotide is 15 to 21 nucleotides in length.

7. The method of claim 5, wherein at least one nucleotide of the oligonucleotide is a nucleotide analogue.

8. The method of claim 5, wherein the oligonucleotide is a gapmer or a mixmer.

9. The method of claim 1, wherein the neurodegenerative condition is a progressive loss of motor function and coordination.

10. The method of claim 9, wherein the condition is spinocerebellar ataxia (SCA).

11. The method of claim 10, wherein the SCA is a polyglutamine SCA, optionally SCA1, SCA 2, SCA3, SCA6, SCA7, or SCA17.

12. The method of claim 10, wherein the SCA is a non-polyglutamine SCAs, optionally SCA4, SCA5, SCA8, SCA9, SCA10, or SCA11 to SCA48.

13. The method of claim 9, wherein the condition is Friedreich's ataxia or ataxia telangiectasia.

14. The method of claim 9, wherein the condition is Huntington's disease spinobulbar muscular atrophy, dentatorubral pallidoluysian atrophy (DRPLA), Parkinson's disease (PD), amyotrophic lateral sclerosis (ALS).

15.-28. (canceled)
